(19) United States
(12) Patent Application Publication
(10) Pub. No.: US 2010/0055107 A1
(43) Pub. Date: Mar. 4, 2010

(54) METHODS FOR PREVENTING HEMATOLOGICAL MALIGNANCIES AND GRAFT VERSUS HOST DISEASE BY ANTI-CD3 PRECONDITIONING

(76) Inventor: Defu ZENG, Arcadia, CA (US)

Correspondence Address: PERKINS COIE LLP, POST OFFICE BOX 1208, SEATTLE, WA, 98111-1208, US

(21) Appl. No.: 12/533,898

(22) Filed: Jul. 31, 2009

Related U.S. Application Data
(60) Provisional application No. 61/085,370, filed on Jul. 31, 2008.
(60) Provisional application No. 61/101,129, filed on Sep. 29, 2008.

(51) Int. Cl. A61K 39/395

(52) U.S. Cl. 424/141.1; 424/130.1

(57) ABSTRACT

Graft versus host disease (GVHD) may be prevented, while preserving a beneficial by graft versus leukemia (GVL) effect, in a subject receiving an allogeneic hematopoietic cell transplant by preconditioning the subject with a CD3 modulator, such as an agonistic anti-CD3 antibody. More than one CD3 modulator may be administered at one time. This method may be performed alone or in conjunction with irradiation of the subject. The use of anti-CD3 to precondition a subject to reduce tissue release of chemokines and prevents tissue dendritic cell migration to draining lymph nodes is also provided. Anti-CD3 preconditioning may also be used to modulate host dendritic cells before hematopoietic cell transplantation.

### PRIORITY CLAIM

This application claims the benefit of U.S. Provisional Patent Application Ser. No. 61/085,370, filed Jul. 31, 2008; and U.S. Provisional Patent Application Ser. No. 61/101,129, filed Sep. 29, 2008; both of which are incorporated herein by reference in their entirety.

### GOVERNMENT INTEREST

The present invention was made with government support by National Institutes of Health Grant No. R01 AI1066008. The government has certain rights in the present invention.

### BACKGROUND

Graft versus host disease (GVHD) is a frequent-occurring and potentially deadly immunological condition in patients who receive an allogeneic hematopoietic cell transplant (HCT). HCTs are varied and may be a bone marrow donation, peripheral blood transplant, cord blood transplant, stem cell transplant, cells differentiated in vitro by various culture methods, or another allogeneic hematopoietic cell transplant. Allogeneic HCTs are used to treat blood-related disorders, certain causes of anemia, and immunological disorders. GVHD occurs when immune cells in the allogeneic (i.e., donated) cell population attack the subject's own cells because the recipient cells are recognized as foreign by the allogeneic cells and an immune response is mounted. GVHD responses may attack any cells, but typically affect the gastrointestinal tract, skin, lungs, and liver (“GVHD target tissues”).

In allogeneic hematopoietic cell transplantation (HCT), both graft versus host disease activity and graft versus leukemia (GVL) activity are predominantly mediated by donor T cells from the allogeneic HCT. Donor T cells are activated in host lymphoid tissues and then migrate to epithelial GVHD target tissues. The graft versus leukemia effect occurs when subjects who have undergone HCT after total body irradiation as treatment for lymphoreticular and hematological malignancies have lower malignancy relapse rates when they also develop GVHD. It would be a significant improvement in the art of treating subjects with these types of malignancies if the negative side-effect of HCT (especially, graft versus host disease) could be reduced or eliminated while maintaining a positive, anti-relapse side effect (graft versus leukemia). In other words, separating GVHD from GVL would significantly benefit HCT-treated subjects.

Alloreactive T cells are activated by the subject's antigen presenting cells (APCs), including dendritic cells (DCs), in secondary lymphoid tissues. DCs in mesenteric lymph nodes (MLN) induce T cell expression of α4β7 and CCR9 receptors that mediate T cell migration to gut tissues because DCs in MLN are able to metabolize vitamin A into retinoid acid (RA) that induces T cells to upregulate α4β7 and CCR9. Similarly, DCs in peripheral lymph nodes induce T cell expression of E-ligand, P-ligand, CCR4, and CCR10 that mediate T cell migration to skin tissue because DCs in peripheral lymph nodes are able to metabolize vitamin D3 to an active form that induces T cell to up-regulate CCR10.

CD103⁺ dendritic cells in lamina propria (LP) may capture antigens locally and then migrate via afferent lymph to draining MLN, where they activate naïve T cells and induce gut tissue-specific homing and chemokine receptors. DC migration from LP to MLN and from dermis to peripheral LN (PLN) both requires DC expression of CCR7 and MLN and PLN DCs reciprocally induce T cell gut and skin tissue tropism. Consistently, DCs in host draining LN have been shown to induce donor T cell expression of gut and skin homing receptors, although donor T cells could still infiltrate GVHD target tissues in recipients deficient in MLN and PLN.

B7H1, also called PD-L1, is a co-inhibitory molecule expressed on resting T cells, B cells, DCs, and macrophages, and is further upregulated upon activation. In addition, B7H1 protein is not usually expressed by tissue parenchymal cells, but can be induced by proinflammatory cytokines such as IFN-γ. Pancreatic islet β cell expression of B7H1 played a critical role in protecting islet β cells in autoimmune NOD mice, in which tissue expression of B7H1 leads to autoreactive T cell energy and reduction of IFN-γ and IL-2 production. B7H1 tolerizes T cells by interaction with its ligand PD-1 on activated T cells. The role of tissue expression of B7H1 in GVHD was previously unknown.

Separating graft versus host disease and graft versus leukemia remains a major challenge in the treatment of hematological malignancies by allogeneic hematopoietic cell transplantation. There remains a need for a more effective method of treating hematological malignancies which includes separating graft versus leukemia from graft versus host disease.

### SUMMARY

In one aspect, a CD3 modulator prevents or reduces the severity of graft versus host disease (GVHD), while preserving the graft versus leukemia (GVL) effect in subjects undergoing allogeneic hematopoietic cell transplantation (HCT) for the treatment of a hematological malignancy. Conditioning of allogeneic recipients with one or more CD3 modulators, such as an agonistic anti-CD3 antibody, other antibody, or agonistic small molecule, prevents GVHD but retains GVL. The other antibody may be anti-CD 28.

The CD3 modulator may be used as a preconditioning agent (meaning administered in advance of HCT), or administered co-extensively with the HCT, or both. The CD3 modulator may be administered alone or in combination with other agents designed to suppress T-cell mediated immune responses, such as corticosteroids (e.g., prednisone). The anti-CD3 antibody or a therapeutically effective fragment thereof may be either monoclonal or polyclonal. The CD3 modulator may administered at any time sufficient to prevent or reduce GVHD before HCT and such time is typically a time period of approximately two weeks to two days before HCT. Of course, if there are multiple administrations of the one or more CD3 modulators, the administrations may vary, and may be start several months in advance and continue until immediately prior to CD3 modulator administration.

The subject may also undergo irradiation before receiving HCT. There may be one or more than one irradiation sessions and the irradiation may be either total body irradiation (TBI) or targeted irradiation. The irradiation may occur before, after, and/or during the administration of the CD3 modulator. When irradiation is administered, it may be administered in conjunction with a B7H1 modulator such as IFN-γ in order to upregulate B7H1 and prevent undesirable damage to tissues. Prevention of GVHD is associated with inhibition of donor T expression of homing and chemokine receptors and inhibition of GVHD target tissue expression of chemokines.

In another aspect, CD3 production and/or activity is controlled via a modulating agent. The CD3 modulating agent may agonize or antagonize any step of the CD3 production and/or activity pathway such that modulating CD3 prevents or reduces in incidence or intensity of GVHD. The CD3 modulating agent may be an anti-CD3 antibody, a therapeutically fragment thereof, or a small molecule. In one aspect, the anti-CD3 antibody is an agonist antibody.

In a further embodiment, CD3 is agonized in conjunction with a second agent that agonizes host T-cell function, wherein such agonizing prevents or reduces in incidence or intensity of hematological malignancies.

A method of inhibiting donor T expression of gut homing α4β7 and chemokine receptor CCR9 by anti-CD3 preconditioning is also provided. In certain embodiments, this inhibition results from reduction of CD103⁺ dendritic cells (DC) in draining mesenteric lymph nodes, which is associated with downregulation of DC expression of CCR7, a receptor required for tissue DC migration to draining LN.

In other aspects, methods are provided for preconditioning with anti-CD3 to reduce not only tissue release of chemokines but also to prevent tissue DC migration to draining LN, and subsequently reducing draining LN DC's capacity in imprinting donor T tissue tropism. Modulation of host DCs by anti-CD3 preconditioning before HCT, which separates GVL from GVHD, is also described.

Another embodiment upregulates tissue parenchymal cell expression of B7H1 induced by donor T-derived IFN-γ to prevent of GVHD in recipients conditioned with a radiation-free anti-CD3-based conditioning regimen. Parenchymal cell expression of B7H1 inhibits memory alloreactive T cell proliferation and expansion. Thus, conditioning procedures and therapies for maintaining or enhancing parenchymal cell expression of co-inhibitory molecules (i.e. B7H1) and methods of administering such co-inhibitory molecules to a subject undergoing HCT are additional embodiments useful for GVHD prevention, which methods have been proven in vivo.

Donor CD8⁺ T cells induced lethal GVHD in anti-CD3-conditioned B7H1^{-/-} BALB/c and unconditioned Rag-2^{-/-} BALB/c recipients, although they induced little signs of GVHD in the B7H1^{+/+} counter parts. Furthermore, when bone marrow (BM) chimeras were used to distinguish the role of B7H1 expressed by hematopoietic cells or by tissue parenchymal cells, the former played mild role and the later played a critical role in GVHD prevention in unconditioned Rag2^{-/-} recipients. In addition, the B7H1 expression by tissue parenchymal cells such as liver was associated with inhibition of donor T cell proliferation, reduction of tissue damage, and prevention of clinical GVHD. The limited role of B7H1 expressed by host hematopoietic cells such as DCs is likely due to the fact that they are rapidly eliminated by donor T cells after HCT, because host DCs were replaced by donor DCs in the HCT recipients about one week after HCT. Therefore, B7H1 expression by host tissue parenchymal cells plays a more important role in GVHD prevention than those expressed by host hematopoietic cells.

Donor T cell-derived IFN-γ was required for induction of host parenchymal cell expression of B7H1 and prevention of GVHD. While wild-type donor CD8⁺ T cells that induced tissue parenchymal cells (i.e. liver) to express B7H1 caused little signs of GVHD in unconditioned Rag2^{-/-} recipients, IFN-γ^{-/-} donor CD8⁺ T cells that failed to induce host parenchymal cells to express B7H1 caused lethal GVHD. In addition, host and donor DCs expressed high levels of B7H1 despite the transplantation with IFN-γ^{-/-} donor CD8⁺ T cells. These results demonstrate that IFN-γ from donor T cells plays a critical role in inducing B7H1 expression in host tissue parenchymal cells but not in DC cells and that parenchymal cell but not DC expression of B7H1 play a critical role in GVHD prevention.

Yet another method of the present invention inhibits donor memory CD8⁺ T cells with B7H1 or another CD8⁺ T modulator. B7H1 expressed by host parenchymal cells inhibited the proliferation and expansion of donor memory T cells in GVHD target tissues. Compared to B7H1 sufficient Rag-2^{-/-} recipients, donor CD8⁺ T cells in B7H1^{-/-} Rag-2^{-/-} recipients expanded stronger in lymphoid tissues early after HCT. However, 25 days after HCT, when de novo developed donor-type CD4⁺ T cells were present in the tissues, donor CD8⁺ T cells in B7H1^{-/-}Rag-2^{-/-} recipients expanded stronger in GVHD target tissues but not in lymphoid tissues. Memory CD8⁺ T cell proliferation can be initiated within peripheral tissues through a tripartite interaction that includes CD4⁺ T cells and recruited dendritic cells. After transplantation of sorted donor CD8⁺ T cells into allogeneic recipients, the alloreactive CD8⁺ T cells interact with host DCs and are activated and expanded in lymphoid tissues early after HCT. At the same time, some of the activated T cells infiltrate GVHD target tissues. Donor CD8⁺ T expansion in lymphoid tissues subsided after host-type DCs are eliminated and replaced with donor-type DCs. In the tissue that expresses B7H1, many donor alloreactive memory CD8⁺ T cells are anergic and less proliferative. In contrast, in the tissues that do not express B7H1, donor alloreactive memory CD8⁺ T cells are less anergic but more proliferative. Therefore, donor memory CD8⁺ T cells expand rapidly in the tissues of B7H1^{-/-} recipients after the presence of de novo developed donor-type CD4⁺ T cells.

It has also been discovered that the second wave expansion of donor memory T cells in TBI-conditioned recipients was associated with lack of expression of B7H1 by GVHD parenchymal tissues such as hepatocytes. Some particular cytokines or the responsiveness of parenchymal cells to cytokines such as IFN-γ may be different in anti-CD3-conditioned or TBI-conditioned recipients, because it was found that, although both anti-CD3 and TBI-conditioning caused cytokine storm with high-level serum IFN-γ, anti-CD3 but not TBI-conditioning upregulated parenchymal tissue expression of B7H1 as demonstrated by B7H1 expression on hepatocytes. Therefore, another embodiment combines the anti-CD3-conditioning regimen with a radiation-free treatment regimen to better maintain the B7H1 expression capacity of host parenchymal tissues. The tissue expression of B7H1 early after HCT in TBI-conditioned recipients contributes to down-regulation of GVHD.

The present methods to prevent GVHD using in anti-CD3-conditioning have many advantages. First, compared to TBI-conditioning alone, anti-CD3-conditioning avoids host tissue damage, so that tissue release of inflammatory chemokines and cytokines is prevented. The use of anti-CD3 conditioning allows for the use of a lower dose of radiation or the elimination of radiation. Second, anti-CD3-conditioning down-regulates host DC's capacity in imprinting donor T expression of tissue homing and chemokine receptors. The reduction of chemokine release by GVHD target tissues and the down-regulation of homing and chemokine receptor expression by donor T cells lead to a marked reduction in donor T migration to GVHD target tissues. Finally, anti-CD3-conditioning well maintains the ability of parenchymal tissues to express co-inhibitory molecules such as B7H1 that tolerizes the infiltrating T cells. Therefore, in anti-CD3-conditioned recipients, even if some alloreactive T cells infiltrate GVHD target tissues, they are tolerized by the B7H1 expressed by parenchymal cells. In contrast, in TBI-conditioned recipients, the capacity of parenchymal cells in expressing B7H1 is damaged and/or overwhelmed by severe infiltration, so that the infiltrating T cells proliferate and expand and cause severe GVHD tissue damage.

These and other advantages are further illustrated and explained with respect to the drawings and in the detailed description.

### BRIEF DESCRIPTION OF THE DRAWINGS

FIG. 1 shows anti-CD3 preconditioning (black bars—anti-CD3+TBI) inhibited liver tissue expression of chemokines as compared to TBI alone (white bars). Five days after HCT, liver tissue expression of Ccl3-5 and Cxcl9-11 was measured with real-time PCR. Mean±SE of 4 replicate experiments is shown.

FIG. 2 shows retinoid acid (RA) induced donor T cell expression of α4β7 and CCR9 during culture with host spleen DCs or MLN DCs from anti-CD3-preconditioned mice. Sorted CD4⁺ T cells (0.2×10⁶) were stimulated with CD11c⁺ DCs (0.1×10⁶) from spleens of BALB/c mice without anti-CD3 preconditioning (FIG. 2A) or MLN DCs from BALB/c mice preconditioned with anti-CD3 (FIG. 2B) in the presence or absence of RA in culture. Four days after culture, donor CD4⁺ T cell expression of α4β7 and CCR9 was measured with flow cytometry. The α4β7⁺ or CCR9⁺ CD4⁺ T cells are gated. One representative of 3 replicate experiments is shown.

FIG. 3 shows anti-CD3 preconditioning changed host DC subset tissue distribution. CD11c⁺ DCs from BALB/c mice with or without anti-CD3 preconditioning were enriched from spleen, MLN, PLN and liver, and their expression of CD103, CD8, CD11b and PDCA-1 (a marker for plasmacytoid DCs) was analyzed with flow cytometry. One representative FACS pattern of 4 replicate experiments and the mean±SE of the percentage of CD103⁺, CD8⁺, CD11b⁺ or PDCA-1⁺ cells among CD11c⁺ DCs are shown.

FIG. 4 shows anti-CD3 preconditioning increased the percentage of Foxp3⁺ Treg cells among donor CD4⁺ T cells. Five days after HCT, spleen cells from BALB/c recipients with or without anti-CD3 preconditioning were stained with anti-H-2^{b}, CD4, TCR-β and Foxp3. FIG. 4A) A representative FACS pattern of 4 replicate experiments. FIG. 4B) Mean±SE of the percentage of Foxp3⁺ CD4⁺ T cells among total donor CD4⁺ T cells.

FIG. 5 shows anti-CD3 but not ATG preconditioning prevented acute GVHD. BALB/c recipients were preconditioned with anti-CD3 (5 μg/g) or ATG (50 μg/g) or control Rat IgG (50 μg/g) on day −9. On day 0, the peripheral blood of the mice was measured for residual T cells, and the mice were then given 800 rads TBI. 6 hours later, the recipients were transplanted with TCD-BM (5×10⁶) and spleen cells (2.5×10⁶) from C57BL/6 donors. Thereafter, the recipients were monitored for clinical signs of GVHD, bodyweight and survival daily. In addition, 4 mice in each group were used for the analysis of CCR7 expression by CD103⁺ DCs in MLN and spleen. FIG. 5A) Before TBI, peripheral blood MNCs from mice preconditioned with anti-CD3 or ATG were stained with TCR-β versus CD4 or CD8, and the TCR-β⁺ CD4⁺ or TCR-β⁺ CD8⁺ T cells were gated. One representative of 12 mice in each group is shown. FIG. 5B) CD11c⁺ DCs from MLN and spleen of recipients preconditioned with anti-CD3 or ATG were stained with antiCD103 and CCR7. CD11c⁺ DCs were shown in CD103 versus CCR7. One representative of 4 replicate experiments is shown. FIG. 5C) Bodyweight changes (mean±SE) of the recipients. There were 8 recipients in each group. FIG. 5D) Survival percentage of recipients.

FIG. 6 shows anti-CD3 preconditioning did not change the host DC's capacity in stimulating donor T cell proliferation and Th1 differentiation. Donor C57BL/6 spleen cells (0.2×10⁶) were stimulated with sorted CD11c⁺ DC cells (0.1×10⁶) from spleen of host BALB/c mice with or without anti-CD3 preconditioning. T cell proliferation was measured with ³H-TdR incorporation 72 hours after culture (FIG. 6A), and percentage of IFN-γ-secreting CD4⁺ and CD8⁺ T cells were measured by intracellular staining 96 hours after culture (FIG. 6B).

FIG. 7A: Peripheral blood T cells were analyzed with flow cytometry nine days after injection. FIG. 7B: Nine days after antibody injection, CD11c⁺ DC-enriched MLN and spleen cells were stained with anti-CD11c, CD103, and CCR7. Gated CD11c⁺ cells were shown in CD103 versus CCR7. CR7⁺ CD103⁺ and CCR7-CD103⁺ cells among total CD11c⁺ cells are gated and their percentages are shown beside the gating boxes. FIG. 7C: Peripheral blood T cell patterns 3 days after anti-CD3 or anti-CD3 (Fab)₂ injection. FIG. 7D: CD11c⁺ DC patterns 3 days after anti-CD3 or anti-CD3 (Fab)₂ injections. One representative of 3 replicate experiments is shown.

FIG. 8 is another experiment (in addition to the experiments show in FIG. 5) demonstrating that anti-CD3 but not ATG preconditioning prevented acute GVHD. BALB/c recipients were preconditioned with anti-CD3 (5 μg/g), ATG (50 μg/g) or control Rat IgG (50 μg/g) on day-9. On day 0, the mice were given 800 rads TBI. 6 hours later, the recipients were transplanted with TCD-BM (5×10⁶) and spleen cells (2.5×10⁶) from C57BL/6 donors. Thereafter, the recipients were monitored for clinical signs of GVHD, bodyweight and survival daily. There were 8 recipients in each group. FIG. 8A) Clinical score (mean±SE) of GVHD of the recipients. FIG. 8B) Bodyweight changes (mean±SE) of the recipients. FIG. 8C) Survival percentage of the recipients.

FIG. 9 shows anti-CD3 preconditioning separated GVL from GVHD in TBI-conditioned recipients. BALB/c mice were preconditioned with anti-CD3 on Day −9. The mice were conditioned with 800 rads sublethal TBI on Day 0. Six hours later, the mice were injected I.V. with TCD-BM cells (5×10⁶) and spleen cells (2.5 or 5×10⁶) from C57BL/6 donors. There were 12 mice in each group combined from 3 replicate experiments. A) Clinical score. B) Survival percentage. C) and D) are images and a graph showing the results of a pathology experiment on liver, skin, and colon tissues from TBI-conditioned recipients with or without anti-CD3 preconditioning. The samples were evaluated for tissue inflammation and damage 60 days after HCT. A representative histopathology and the mean±SE of 6 recipients in each group are shown. E), F) and G) Anti-CD3-preconditioned BALB/C recipients were injected I.V. with BCL1 cells transfected with luciferase (Luc⁺), and donor TCD-BM and spleen cells (2.5×10⁶). There were 8 mice in each group combined from 2 replicate experiments. The survival percentage (FIG. 9E), representative photos of in vivo bioluminescent imaging of Luc⁺ BCL1 cells (FIG. 9F), and the intensity (photo/second) of BLI (FIG. 9G) are shown.

FIG. 10 shows anti-CD3 preconditioning inhibited donor T cell infiltration of GVHD target tissues. Five days after injection of donor TCD-BM and spleen cells (5×10⁶), the percentage and yield of donor T cells in spleen, MLN, PLN, liver, gut and skin of the recipients with or without anti-CD3 preconditioning were compared. There were 4 recipients in each group. FIG. 10A shows a representative FACS pattern. Mononuclear cells from different tissues were stained with anti-TCRαβ versus anti-H-2^{b} (donor MHC I), and the donor-type T cells were gated. FIG. 10B is a graph showing the means result plus or minus the standard error (“mean±SE”) of the donor T cell percentage among total mononuclear cells of 4 recipients. FIG. 10C is a graph showing the mean±SE of the yield of donor T cells in different tissues. FIG. 10D is a representative intracellular IFN-γ staining pattern of the gated H-2^{b+} CD4⁺ or H-2^{b+}CD8⁺ T cells. The IFN-γ⁺ cells were gated. FIG. 10E is a graph showing the mean±SE of the percentage of donor IFN-γ⁺ CD4⁺ or CD8⁺ cells of 4 examined recipients. FIGS. 10F & 10G are bar graphs showing the mean±SE of serum IFN-γ and TNF-α levels of 6 examined recipients.

FIG. 11 shows anti-CD3 preconditioning inhibited donor T cell expression of homing and chemokine receptors. Five days after HCT, donor T expression of gut homing α4β7 and CCR9 receptors in MLN, donor T expression of skin homing E-Ligand, P-Ligand, CCR4 and CCR10 in PLN, and donor T expression of non-tissue specific CCR5 and CXCR3 chemokine receptors in spleen were compared. There were 4 recipients in each group. FIG. 11A is a representative FACS pattern of CCR9 and α4β7 by gated H-2b⁺CD4⁺ or H-2b⁺CD8⁺ donor T cells from MLN, as well as representative FACS pattern of CCR4, CCR10, E-Lig, and P-Lig of donor CD4⁺ or CD8⁺ T cells from PLN. FIG. 11B is a graph showing the mean±SE of CCR9⁺ or α4β7⁺ cells among donor CD4⁺ or CD8⁺ T cells from MLN. FIG. 11C is a graph showing the mean±SE of CCR4⁺, CCR10⁺, E-Lig⁺ or P-Lig⁺ cells among donor CD4⁺ or CD8⁺ T cells from PLN. FIG. 11D is a representative FACS pattern of CCR5 and CXCR3 by gated H-2b⁺CD4⁺ or H-2b⁺CD8⁺ donor T cells from spleen. FIG. 11E shows two bar graphs with the mean±SE of CCR5⁺ or CXCR3⁺ cells among gated donor CD4⁺ or CD8⁺ T cells from spleen of 4 recipients.

FIG. 12 shows anti-CD3 preconditioning inhibited GVHD target tissue expression of chemokines. Expression of chemokine mRNA at day 5 after HCT in various tissues (including skin and colon) of TBI-conditioned recipients with or without anti-CD3 preconditioning was measured by real-time PCR. FIG. 12A) Expression of Ccl17, Ccl22, Ccl27 and Ccl28 by skin tissues. FIG. 12B) Expression of Ccl25, Ccl3-5 and CXCL9-11 by colon tissue. Data were presented relative to the expression in the syngeneic control recipients. Mean±SE of 4 recipients in each group is shown.

FIG. 13 shows anti-CD3 preconditioning reduced CD103⁺ DCs in MLN and reduced MLN DC capacity in induction of donor T cell expression of α4β7 and CCR9. Spleen and MLN cells of BALB/c mice with or without anti-CD3 preconditioning were harvested and enriched for CD11c⁺ DCs by micromagnetic beads. The CD11c⁺ enriched cells were further analyzed with flow cytometry or used for in vitro culture. FIG. 13A: A representative FACS pattern of CD103 expression among CD11c⁺ DCs. FIG. 13B: Mean±SE of CD103⁺ cells among CD11c⁺ DCs and the yield of CD103⁺ CD11c⁺ DCs in spleen and MLN of 4 mice with or without anti-CD3 preconditioning. FIG. 13C: Sorted CD4⁺/CD8⁺ T cells (0.2×10⁶) from C57BL/6 spleen were co-cultured with enriched CD11c⁺ DCs (0.1×10⁶) from the MLN of host BALB/c mice with or without anti-CD3 preconditioning for 4 days. Thereafter, donor CD4⁺ or CD8⁺ T cells were analyzed for the expression of α4β7 and CCR9. One representative of 4 replicate experiments is shown. FIG. 13D: Mean±SE of the percentage of α4β7⁺ or CCR9⁺ cells among donor CD4⁺ or CD8⁺ T cells in the culture of the 4 experiments. FIG. 13E: Sorted donor CD8⁺ T cells (0.2×10⁶) were co-cultured with CD103⁺ DCs (0.05×10⁶) from MLN and spleen of the host mice, and then donor CD8⁺ T cell expression of α4β7 and CCR9 were analyzed. The α4β7⁺ or CCR9⁺ CD8⁺ T cells were gated. One representative of 4 replicate experiments is shown. FIG. 13F: Mean±SE of the α4β7⁺ or CCR9⁺ cells among donor CD8⁺ T cells of the 4 experiments.

FIG. 14 shows anti-CD3 preconditioning downregulated CCR7 expression by CD103⁺ DCs in intestine LP and MLN, and this effect required anti-CD3 activation of host T cells. FIG. 14A: Nine days after anti-CD3 preconditioning, CD11c⁺ DCs from LP, MLN and spleen of the BALB/c mice with or without preconditioning were analyzed with flow cytometry. The gated CD11c⁺ DCs are shown in CD103 versus CCR7. The percentage of CCR7⁺ CD103⁺ or CCR7⁻CD103⁺ cells among total DCs is shown beside the gating boxes. One representative of 4 replicate experiments is shown. The mean±SE of the percentage of total CD103⁺ DCs among total CD11c⁺ DCs in different tissues before and after anti-CD3 preconditioning is 85.4±2.9 versus 72.9±6.5 (LP), 57.2±1.8 versus 8.4±0.8 (MLN), and 7.1±0.8 versus 28.9±3.1 (spleen). FIG. 14B: Mean±SE of CCR7⁺ cells among CD103⁺ DCs. FIG. 14C: Mean±SE of CCR7 expression level (mean fluorescence) by CD103⁺ DCs. FIG. 14D: T cell deficient Nu/Nu mice and IFN-γ^{-/-} mice as well as wild-type mice were preconditioned with anti-CD3 or PBS, 9 days later, the MLN cells were enriched with CD11c⁺ DCs and the percentage of CD103⁺ DCs among total CD11c⁺ DCs was measured. One representative of 4 examined mice in each group is shown. FIG. 14E: Mean±SE of the percentage of CD103⁺ DCs in MLN of 4 recipients with or without anti-CD3 preconditioning.

FIG. 15 shows GVHD induced in B7H1^{-/-} but not wild-type recipients conditioned with anti-CD3. B7H1^{-/-} and Wild-type BALB/c mice were conditioned with anti-CD3 on Day −7. The mice were injected I.V. with CD8⁺ T cells (20×10⁶) and BM cells (100×10⁶) from C57BL/6 donors on Day 0. The CD8⁺ T cells were injected again 5 days after first injection. There were 12 mice in each group, combined from 3 replicate experiments. FIG. 15A: Clinical score; FIG. 15B: Percentage of body weight change; FIG. 15C: Percentage of survival; FIG. 15D: Representative of H&E staining of colon, liver, lung, and skin tissue sections of recipients 100 days after HCT; FIG. 15E: Mean±SE of tissue pathology score; N=4.

FIG. 16 shows parenchymal cells expression of B7H1 was associated with donor T cell anergy. 13-25 days after HCT, hepatocytes were harvested for evaluation of B7H1 expression. Donor T cells in spleen and liver were harvested for proliferation and cytokine secretion assays. FIG. 16A: Hepatocytes mRNA level expression of B7H1. Representative of 3 replicate experiments is shown. FIG. 16B: Anti-B7H1 or Rat IgG staining of hepatocytes surface B7H1 before and after HCT at different time points. Rat IgG control staining is shown as gray area. Representative FACS pattern of 4 replicate experiments is shown. FIG. 16C: Paraffin liver sections were immunohistochemical stained for B7H1, day 20 after HCT or before HCT. Representative microscopic images are shown of 4 replicate experiments. FIG. 16D: Mean±SE of yield of donor CD8⁺ T cells in spleen and liver of wild-type and B7H1^{-/-} recipients 20 days after HCT, N=4. FIG. 16E: Twenty days after HCT, donor CD8⁺ T cells in spleen and liver were isolated and stimulated with plate-bound anti-CD3 and anti-CD28 in culture medium for 72 hours. H3-TdR (1 μci/well) was added to the culture 12 hrs before harvest. Mean±SE of triplicate culture and representative of three replicate experiments are shown. FIG. 16F: Percentage and yield of donor IFN-γ⁺CD8⁺ T cells in recipients' liver 20 days after HCT. Left panel: representative intracellular staining pattern of 4 replicate experiments. Right panel: yield of IFN-γ⁺ donor CD8⁺ T cells.

FIG. 17 shows tissue expression of B7H1 was associated with GVHD prevention in unconditioned Rag2^{-/-} recipients. Unconditioned Rag2^{-/-} and B7H1^{-/-}Rag2^{-/-} mice were injected i.v. with CD8⁺ T cells (20×10⁶) TCD-BM cells (2.5×10⁶) from C57BL/6 donors. The recipients were monitored for clinical GVHD twice a week. There were 12 mice in each group, combined from 3 replicated experiments. FIG. 17A: Clinical score; FIG. 17B: Body weight change; FIG. 17C: Survival percentage; FIG. 17D: At different time points after HCT, hepatocytes were harvested for analysis of expression of B7H1 with flow cytometry. Representative of 4 replicate experiments is shown. FIG. 17E: Paraffin liver sections were immunohistochemical stained for B7H1, 13 days after HCT or before HCT. Representative microscopic images of 4 replicate experiments is shown.

FIG. 18 shows donor T-derived IFN-γ was required for upregulation of hepatic B7H1 and prevention of GVHD in unconditioned Rag2^{-/-} mice. Rag2^{-/-} recipients were transplanted with CD8⁺ T cells (20×10⁶) and TCD-BM cells from IFN-γ^{-/-} C57BL/6 donors. FIG. 18A: Clinical score; FIG. 18B: Body weight change; FIG. 18C: Survival percentage; FIG. 18D: Representative hematoxylin and eosin (H&E) staining of colon, liver, lung, and skin sections of recipients 40 days after HCT; FIG. 18E: Mean±SE of histopathology score; n=6; FIG. 18F: B7H1 expression on hepatocytes and CD11c⁺ DCs was measured by Flow Cytometry in Rag2^{-/-} recipients given WT or IFN-γ^{-/-} donor CD8⁺ cells 20 days after HCT. Representative FACS pattern of 4 replicate experiments is shown.

FIG. 19 shows B7H1 expression by host tissue parenchymal cells but not hematopoietic cells played a critical role in GVHD prevention. FIG. 19A: Various chimeras were generated by reconstitution of lethally irradiated Rag^{-/-} or B7H1^{-/-}Rag2^{-/-} mice with BM cells from either Rag2^{-/-} or B7H1^{-/-}Rag2^{-/-} mice. The phenotype of the chimeras is shown. FIG. 19B shows the clinical score and FIG. 19C shows the survival percentage of chimeras after being transplanted with CD8⁺ T cells (20×10⁶) and TCD-BM (2.5×10⁶) from C57BL/6 donors.

FIG. 20 shows B7H1 deficiency augmented donor CD8⁺ T cell proliferation and expansion in unconditioned Rag2^{-/-} recipients. FIG. 20A: Representative of in vivo BLI of recipients. Unconditioned Rag2^{-/-} or B7H1^{-/-}Rag2^{-/-} mice were injected with donor CD8⁺ T cells (20×10⁶) from luciferase transgenic C57BL/6 combined with TCD-BM (2.5×10⁶) from WT C57BL/6 mice. FIG. 20B: Kinetic curve of total body photon emission/second in Mean±SE of 6 mice in each group. FIG. 20C: Representative FACS patterns of donor-type H2 k^{b+} TCRαβ⁺, CD4⁺, CD8⁺ cells among total blood mononuclear cells from recipients on days 13 and 30 after HCT. There are 4 mice in each group. FIG. 20D: Donor CD8⁺ T cell proliferation in lymphoid organ and GVHD target tissues from Rag2^{-/-} or B7H1^{-/-}Rag2^{-/-} mice was compared by in vivo BrdU labeling. Mice were i.p injected with BrdU on day 30 after HCT. Lymphocytes from various tissues were stained for donor-type H2k^{b+} CD8 and BrdU. Representative FACS pattern of 4 replicate experiments is shown. FIG. 20E: Yield of BrdU⁺ CD8⁺ T cells, N=4.

FIG. 21 shows the results of wild type mice treated with anti-CD3 before total body irradiation as opposed to control wild type mice not treated before TBI. FIG. 21A are images of representative in vivo recipients of each type and a graph showing photons/sec(×10⁻⁶) as a function of days after hematopoetic cell transplantation for WT-TBI and WT-CD3 mice. FIG. 21B: At different time points after HCT, hepatocytes were harvested for analysis of expression of B7H1 with flow cytometry.

### DETAILED DESCRIPTION

Graft versus host disease (GVHD) may be prevented and separated from graft versus leukemia (GVL) effect in a subject receiving an allogeneic transplant by administering a therapeutically effective amount of a CD3 modulator to a subject. The CD3 modulator may be administered before, during, and/or after the transplant in any combination of: .i before only, ii. before and during, or iii. before, during, and after, but is preferably administered before the transplant (“preconditioned with a CD3 modulator”). The CD3 modulator is an anti-CD3 antibody or therapeutically effective fragment thereof in certain embodiments.

A “CD3 modulator” modulator as used herein includes any anti-CD3 antibody, any anti-CD3 antibody fragment or variant thereof, or any CD3 small molecule modulator that, at a minimum, possesses the ability to inhibit CD3 or prevent one or more of the effects of an immunological reaction to allogeneic cells or tissue. Regarding the anti-CD3 antibodies, the references cited herein discuss creation of the antibodies or fragments or variants thereof as discussed below. In addition, any of multiple procedures known in the art may be used for the production of polyclonal or monoclonal antibodies to CD3, a derivative, or analog thereof. For the production of antibody, various host animals can be immunized by injection with the native CD3 protein, or a synthetic version, or derivative (e.g., fragment) thereof, including but not limited to rabbits, mice, rats, etc. Various adjuvants may be used to increase the immunological response, depending on the host species, and including but not limited to Freund's (complete and incomplete), mineral gels, peptides, and the like. For preparation of monoclonal antibodies directed toward a CD3 protein sequence or analog thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Monoclonal antibodies can be produced in germ-free animals (PCT/US90/02545) or “chimeric antibodies” may also be used (Morrison et al., 1984, Proc. Natl. Acad. Sci. U.S.A. 81:6851-6855; Neuberger et al., 1984, Nature 312:604-608; Takeda et al., 1985, Nature 314:452-454).

A “therapeutically effective” amount of the CD3 modulator as used herein is an amount of an agent that is sufficient to prevent the onset of or to reduce the severity of GVHD. The therapeutically effective amount will vary with the age and physical condition of the patient, body weight, the severity of the condition of the patient being treated, the duration of the treatment, the nature of any concurrent treatment, the pharmaceutically acceptable carrier used, the source of the transplanted material, whether and when the transplantation occurred and other factors that can be weighed and considered by those of skill in the art, who may be scientists and/or treating physicians. A therapeutically effective amount may be determined by in vitro of in vivo methods. A typical daily dosage may range from about 0.0001 mg/kg to up to 100 mg/kg or more, depending on the factors mentioned above.

“Pharmaceutically acceptable carriers” are preferably solutions for injection, but may also be solid dosage forms such as tablets or capsules, liquid preparations for oral administration, powders or other formulations for inhalation, or any other acceptable carriers, the selection of which are known in the art.

As used herein, the term “anti-CD3 preconditioning” refers to use of or treatment with a CD3 modulator, such as a CD3 agonist, prior to the subject receiving transplantation of hematopoietic cells. An exemplary “anti-CD3” preconditioning regimen includes administering or treating an individual suffering from a hematological malignancy, a therapeutically effective amount of one or more antibody specific to CD3, wherein the CD3 antibody is administered prior to the individual receiving cell transplantation, and optionally, also prior to receiving irradiation. The treatment with CD3 antibody thus preconditions the individual prior to his or her receiving allogeneic transplantation therapy.

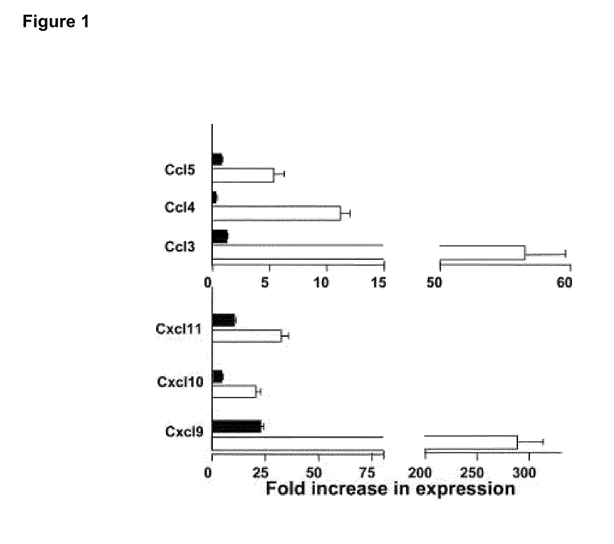

In order to test whether separation of GVL activity from GVHD by anti-CD3 conditioning occurs via modulation of host DCs, the GVL activity was compared to GVHD activity in TBI-conditioned recipients with or without anti-CD3 preconditioning. Then, tissue distribution of DC subsets and their capacity in imprinting donor T tissue tropism in draining lymph node of skin and gut, as well as GVHD target tissue expression of chemokines, was compared. The results were that preconditioning with anti-CD3 prevented GVHD but retained GVL in TBI-conditioned recipients by inhibition of donor T cell migration to GVHD target tissues, which was associated with a marked reduction in donor T expression of homing and chemokine receptors and GVHD target tissue expression of chemokines. (See, for example, FIG. 1 showing anti-CD3 preconditioning inhibited chemokine expression in liver tissue.) Furthermore, the reduction of T cell expression of gut homing and chemokine receptors was due to a reduction of CD103⁺ DCs in MLN, which was associated with LP DC downregulation of CCR7 after anti-CD3 preconditioning. Thus, anti-CD3 preconditioning can modulate host DC subset tissue distribution and inhibit donor T migration to GVHD target tissues.

Anti-CD3 preconditioning markedly reduced donor T cell expression of α4β7 and CCR9 in MLN and markedly reduced T cell expression of P-ligand, E-ligand, CCR4, and CCR10 in PLN. Anti-CD3 preconditioning also markedly reduced the expression of CCL25 (CCR9 ligand) in gut, and reduced the expression of CCL17 and CCL22 (CCR4 ligand), CCL27 and CCL28 (CCR10 ligand) in skin. In addition, the expression of other chemokines that regulate Th1 cell migration into inflammatory tissues, including CCL3-5 (CCR5 ligands), CXCL9-11 (CXCR3 ligands) were also markedly reduced in gut and skin tissues after anti-CD3 preconditioning. Therefore, anti-CD3 preconditioning prevents donor T infiltration of gut and skin tissues.

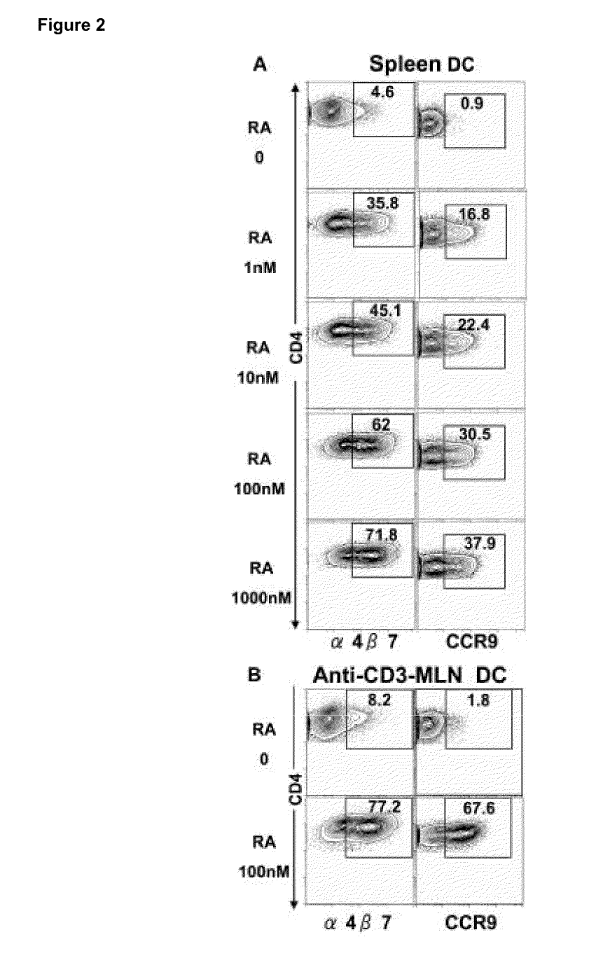

The reduction of donor T cell expression of gut homing and chemokine receptors by anti-CD3 preconditioning results from reduction of CD103⁺ DCs in draining MLN. CD103⁺ DCs in MLN induced T cells express gut homing α4β7 and chemokine receptor CCR9. Only the CD103⁺ DCs from MLN but not those from spleen were able to induce donor T cell expression of α4β7 and CCR9, especially CCR9, due to the difference in their capacity in producing retinoic acid (RA). Addition of RA to the culture led to the induction of donor T expression of CCR9 by spleen DCs as well as by MLN DC from anti-CD3-preconditioned mice (FIG. 2). This was consistent with the fact that reduction of CD103⁺ DCs in MLN led to a reduction of α4β7⁺ or CCR9⁺ donor T cells in the recipient, even when there was an increase of CD103⁺ DCs in the spleen. RA is required for induction of T cell expression of gut homing receptors, and MLN DCs were able to secret RA by metabolizing Vitamin A.

The reduction of CD103⁺ DCs in MLN was associated with down-regulation of CCR7 expression by CD103⁺ DCs in intestine LP after anti-CD3 preconditioning. CD103⁺ DCs in LP carried the orally administered OVA peptide to MLN and induced the antigen-specific OT-II T cells to express α4β7 and CCR9, and the CD103⁺ DC migration from intestinal LP to draining MLN was CCR7-dependent. The marked reduction of CD103⁺ DCs in MLN or marked increase of CD103⁺ DCs in spleen after anti-CD3 preconditioning was associated with a significant reduction of CCR7 expression of CD103⁺ DCs in intestinal LP. These changes were dependent on anti-CD3 activation of host T cells, although IFN-γ was not required.

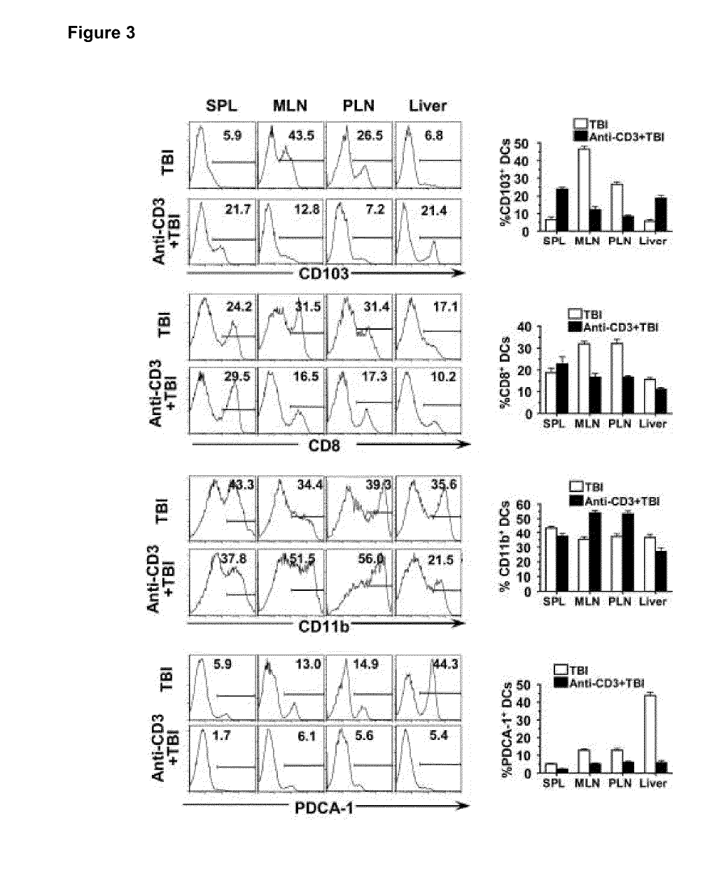

Anti-CD3 preconditioning markedly reduced chemokine expression in GVHD target tissues such as gut, skin, and liver in TBI-conditioned recipients, which results from the reduction of serum levels of IFN-γ and TNF-α as well as the reduction of some DC subsets such as plasmacytoid DCs in the tissues, because plasmacytoid DC cells were the initiators of a complex chemokine and cytokine network. A marked reduction of PDCA-1⁺ plasmacytoid DC in liver tissues after anti-CD3 preconditioning was observed (FIG. 3, bottom row), which may subsequently reduce tissue release of chemokines. Anti-CD3 preconditioning both downregulates donor T expression of chemokine receptors and downregulates chemokine release by GVHD target tissues. Accordingly, donor T cell infiltration of GVHD target tissues is prevented, although they are still activated and able to kill host hematological cells and tumor cells in lympho-hematological tissues.

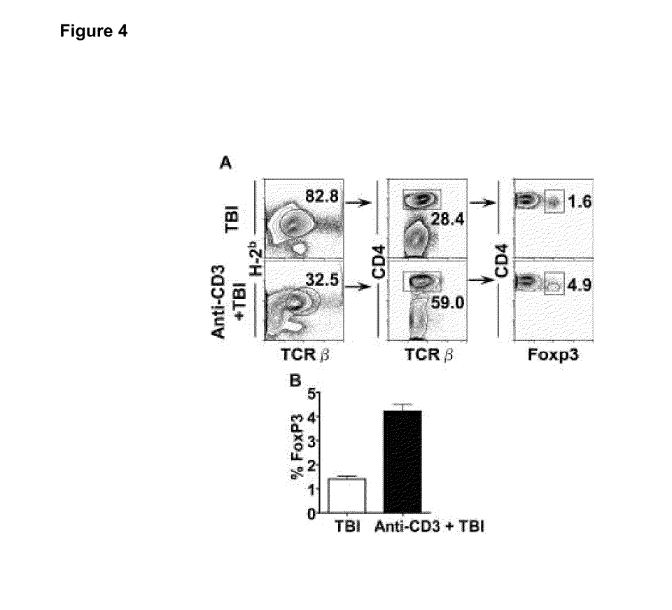

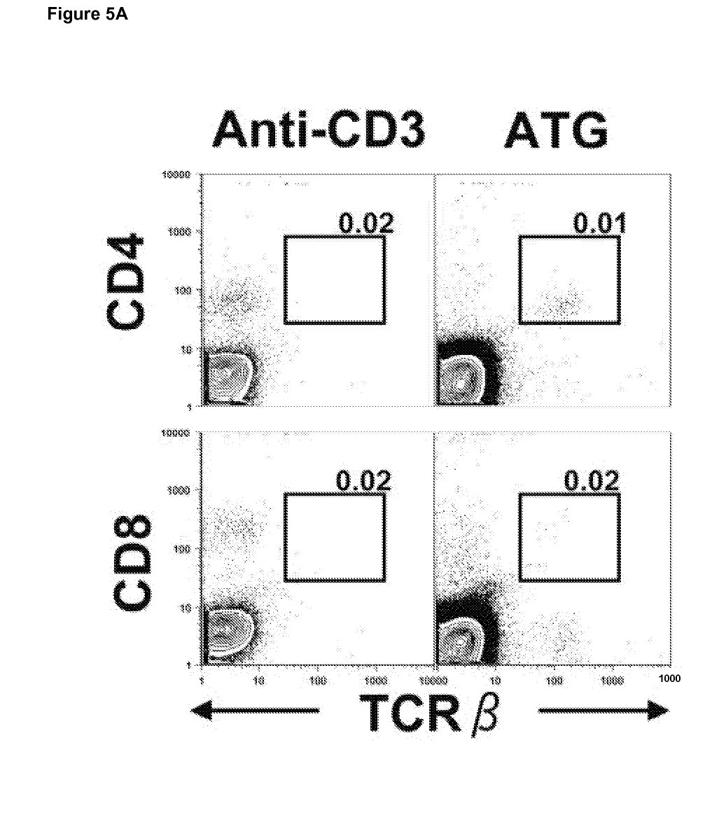

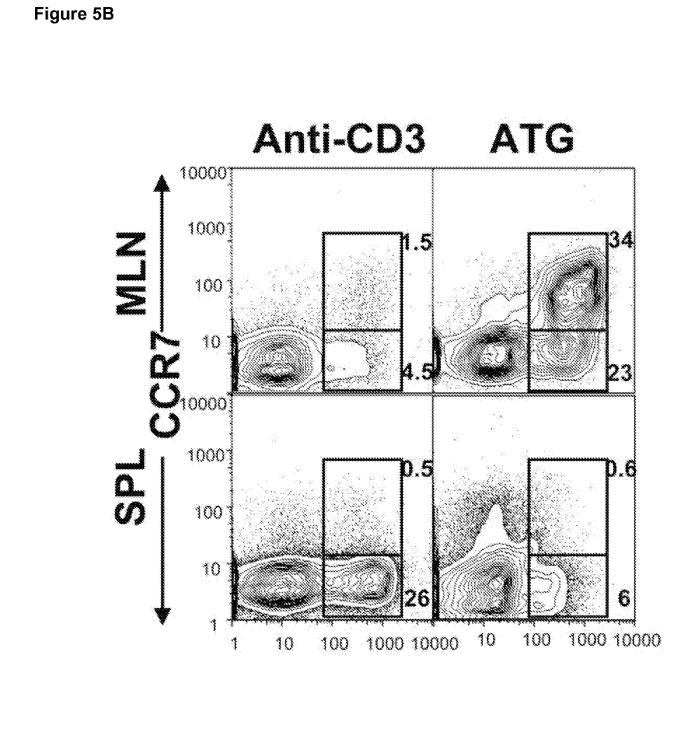

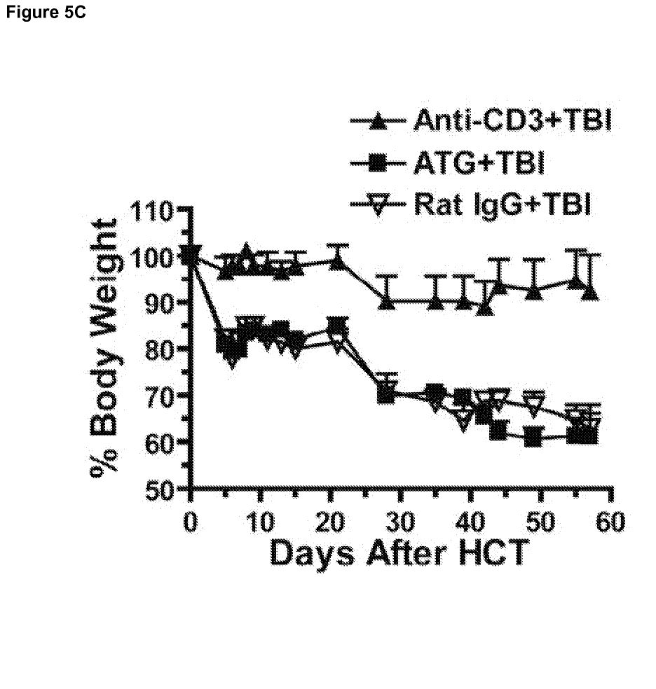

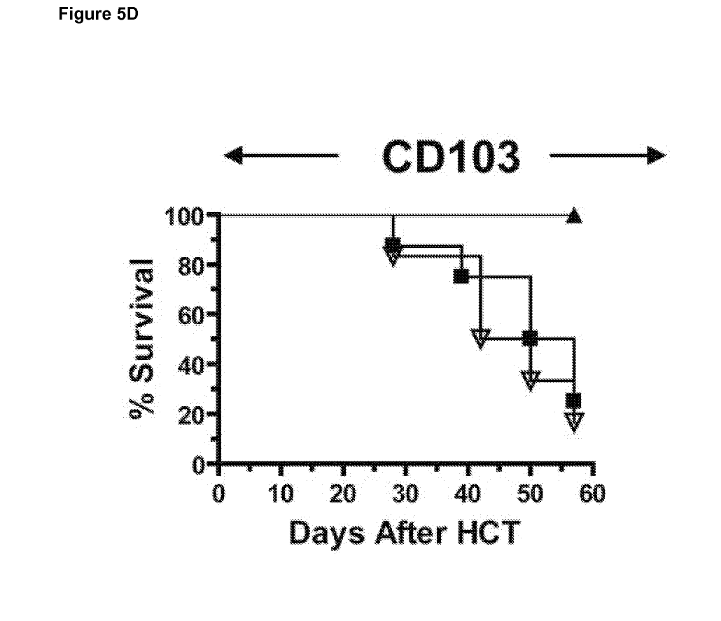

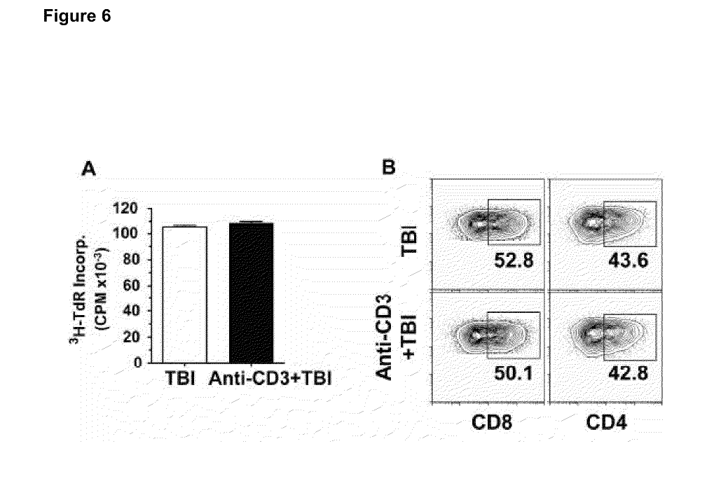

Anti-CD3 treatment induced expansion of FoxP3⁺ Treg cells in anti-CD3-treated mice and a 3-fold increase in donor FoxP3⁺CD4⁺ T cells in recipients preconditioned with anti-CD3 was observed (FIG. 4). The increased donor Treg cells contribute to the inhibition of donor T proliferation and Th1 differentiation in the anti-CD3-preconditioned recipients and prevent GVHD. FIG. 5 shows anti-CD3 but not ATG preconditioning prevented acute GVHD. Sorted DCs from recipients with or without anti-CD3 preconditioning showed similar capacity in stimulating donor T cell proliferation and differentiation into Th1 (FIG. 6). The reduction of donor T cell yield in lymphoid tissues was about 2-fold and the reduction of donor T cell infiltration in gut and skin was more than 15-fold.

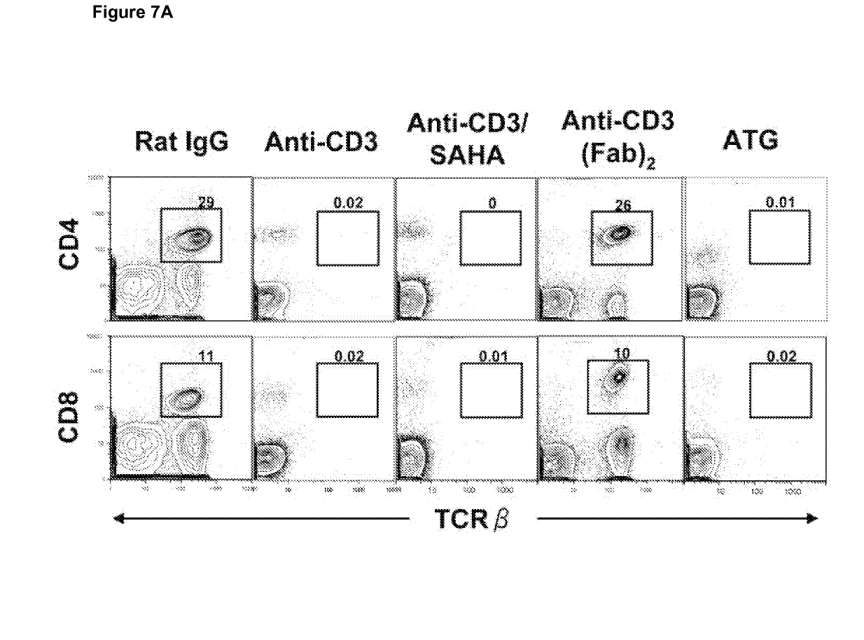

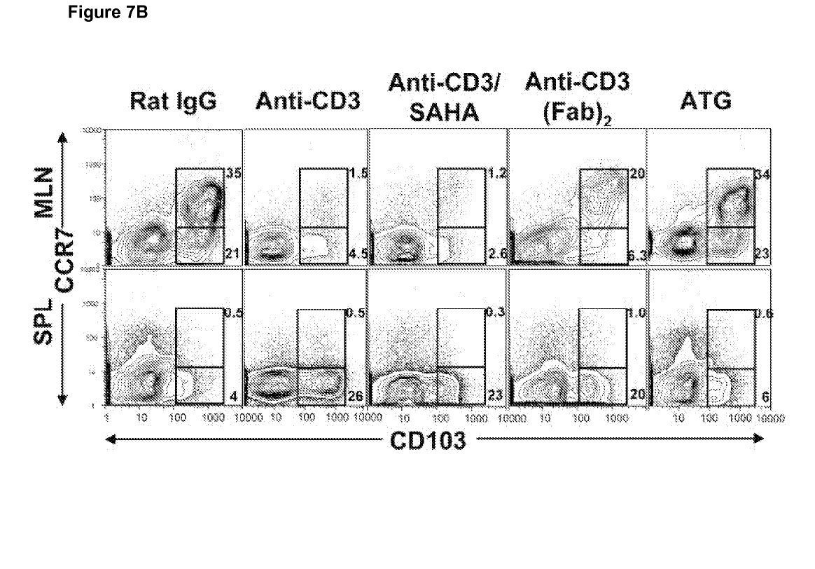

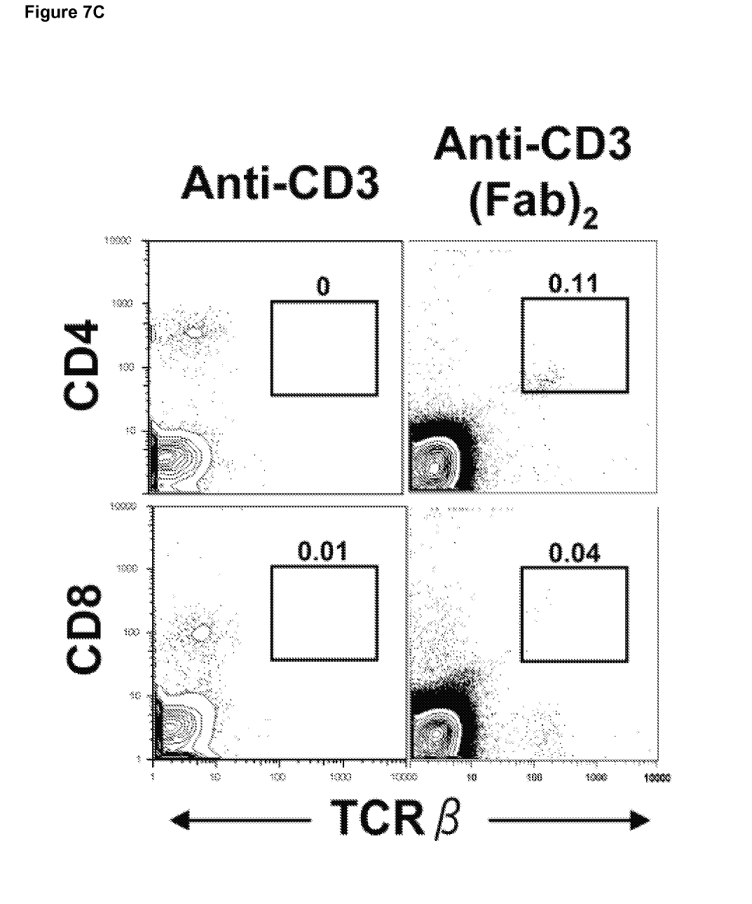

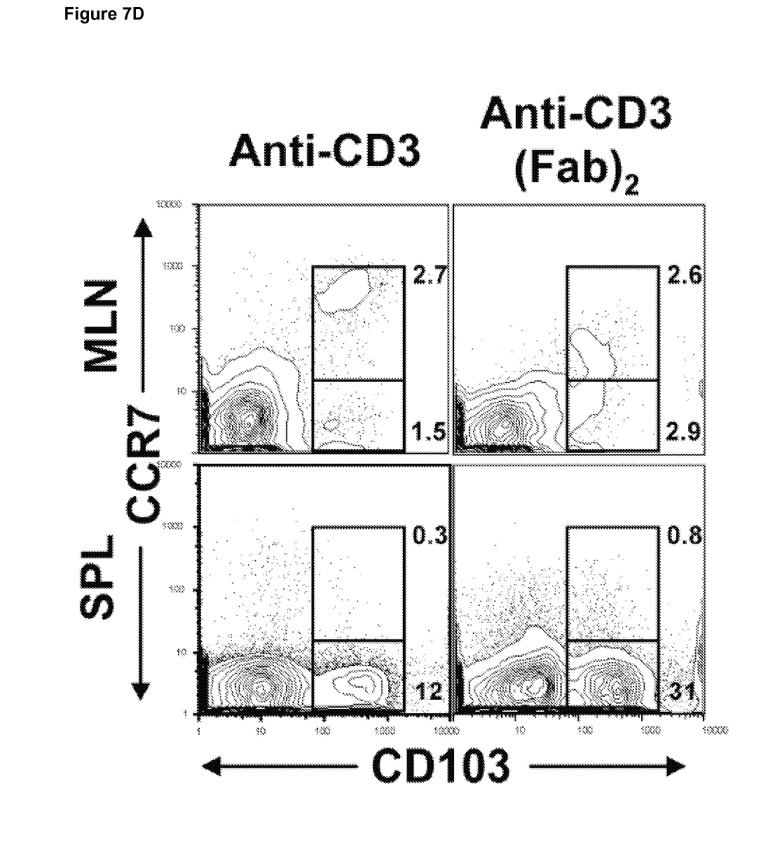

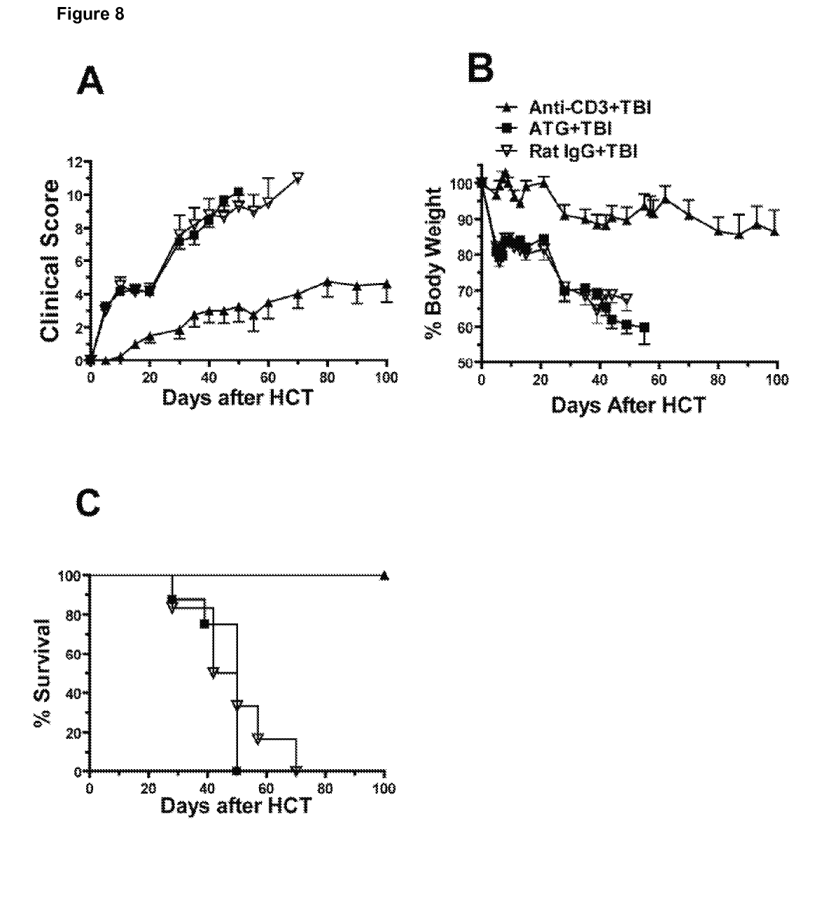

Modulation of DC subset tissue distribution before HCT by CD3 modulators, such as anti-CD3 agonistic antibodies, leads to confinement of donor T cells to lymphohematological tissues, prevents tissue DC migration to draining LN, and separates GVL from GVHD. When anti-CD3 was administered 9 days before HCT, anti-CD3 was not detectable in the recipient serum by the time that the donor T cells were injected, so the anti-CD3 had little direct effect on donor T cell activation or GVL activity. Although anti-CD3-preconditioning modulated host DC and prevented GVHD, preconditioning with anti-thymocyte globulin (ATG), a clinically used reagent for reduced intensity conditioning (RIC), did not reduce CCR7⁺CD103⁺ DCs in host MLN or prevent GVHD in TBI-conditioned recipients (FIGS. 7 & 8), which explains why patients conditioned with ATG-based RIC still developed severe GVHD.

Any issue of a cytokine storm triggered by anti-CD3 when intact anti-CD3 is used may be avoided by using anti-CD3 (Fab)² or histone deacetylase inhibitor SAHA. Preconditioning with anti-CD3 (Fab)₂ or with anti-CD3 and SAHA modulated host DCs, which was similar to preconditioning with intact anti-CD3 alone. FIG. 7 shows anti-CD3, anti-CD3/SAHA, anti-CD3 (Fab)₂ but not ATG preconditioning modulated host DC tissue distribution. Modulation of DC tissue distribution via anti-CD3-preconditioning was in association with host T cell activation, which was associated with down-regulation of TCRαβ receptor and depletion of TCRαβ⁺ T cells in peripheral blood. Therefore, the effects of anti-CD3, anti-CD3/SAHA, anti-CD3 (Fab)₂, and ATG on activation of host peripheral blood T cells were tested. Accordingly, BALB/c recipient mice were injected with anti-CD3 (5 μg/g), anti-CD3 (5 μg/g) and SAHA (40 μg/g), anti-CD3 (Fab)₂ (5 μg/g), ATG (50 μg/g) or control Rat IgG (50 μg/g). The CD4⁺ and CD8⁺ TCRαβ⁺ T cells were checked on days 3, 5, 7, and 9 after antibody injection.

The TCRαβ⁺ T cells in blood were not detectable from days 3 to days 9 after injection of anti-CD3, 3 anti-CD3/SAHA or ATG. However, TCRαβ⁺ T cells in blood of anti-CD3 (Fab)₂ treated mice gradually came back, and reached close to normal level by day 9, although it was not detectable on day 3 (see FIGS. 7 A and C). The DC subset change was first compared on day 9. Although anti-CD3 and anti-CD3/SAHA treatment markedly reduced CCR7⁺CD103⁺ DCs in MLN, anti-CD3 (Fab)₂ only partially reduced the CCR7⁺CD103⁺ DCs in MLN, and ATG treatment did not reduce CCR7^{+CD}103⁺ DCs in MLN at all (see FIG. 7B). Since the peripheral blood T cells were not detectable by day 3, but came back already by day 9 after anti-CD3 (Fab)₂ injection, the DC subset change in MLN of mice treated with anti-CD3 or anti-CD3 (Fab)₂ was compared again by day 3 after injection, and anti-CD3. (Fab)₂ treatment also markedly reduced CCR7⁺CD103⁺ DCs in MLN 3 days after treatment (see FIG. 7D).

Therefore, preconditioning with the clinically applicable FCR-non-binding anti-CD3 (as set forth in Blazar, et al. J. Immunol. 1997; 159:5821-5833) will modulate host DCs and separate GVL from GVHD. Modulation of HCT recipients with CD3-specific antibodies before HCT presents a new approach for separation of donor T cell mediated GVL from GVHD.

Tissue parenchymal cell expression of B7H1 was found to play a critical role in GVHD prevention in anti-CD3-conditioned recipients and unconditioned Rag2^{-/-}. In addition, parenchymal cell expression of B7H1 results in donor T anergy (i.e., lack of responsiveness to an antigen) and reduction of IFN-γ production, as well as inhibition of memory T cell expansion in GVHD target tissues.

In a major histocompatability complex (MHC)-mismatched hematopoietic cell transplantation model of C57BL/6 donor to BALB/c recipient, donor CD8⁺ T cells induce little signs of GVHD in anti-CD3-conditioned wild-type recipient and unconditioned Rag2^{-/-} recipients. In contrast, they induce severe lethal GVHD in the B7H1^{-/-} counter parts. Furthermore, donor CD8⁺ T cells induce mild GVHD in chimeric Rag2^{-/-} recipients with B7H1 deficiency in hematopoietic cells but severe lethal GVHD in Rag2^{-/-} recipients with B7H1 deficiency in tissue parenchymal cells. In addition, parenchymal cell expression of B7H1 is dependent on donor T-derived IFN-γ, which results in donor T cell anergy and reduction of IFN-γ production, as well as inhibition of memory T cell expansion in GVHD target tissues. Therefore, tissue parenchymal cell expression of B7H1 play an important role in GVHD prevention, and the radiation-free anti-CD3-based conditioning regimen may better maintain this protective mechanism.

The following examples further illustrate the novel methods and uses for CD3 modulators, such as anti-CD3 agonist antibodies, and B7H1 in preventing hematological malignancies, such as GVHD, as described herein.

### Example 1

### Materials and Methods

Mice: C57BL/6 (H-2^{b}), BALB/c (H-2^{d}), and Rag2^{-/-}BALB/c (H-2^{d}) mice were purchased from NCI Laboratories (Frederick, Md.). The luciferase transgenic (luc⁺) C57BL/6 and B7H1^{-/-} BALB/c mice were generated as previously described.^{9,18} B7H1^{-/-}Rag2^{-/-} mice were generated by back-crossing B7H1^{-/-} BALB/c to the Rag2^{-/-} BALB/c mice. All animals were maintained in a pathogen-free room at City of Hope Research Animal Facility. Male mice at an age of 6-12 wk were used in the current studies. Animal use protocols were approved by the institutional review committee.

Conditioning of recipients and HCT: Production of anti-CD3 mAb (145-2C11) was described in a previous publications.⁹¹ Recipient BALB/c mice were injected i.v. with anti-CD3 (5 μg/g) on day −9 and were given sublethal total body irradiation (TBI, 800 rads) on day 0, then, the recipients were transplanted with donor TCD-BM cells (5×10⁶) and whole spleen cells (2.5˜5×10⁶). For GVL experiments, Luc⁺ BCL1 cells (0.5×10⁶) were intraperitoneally injected at the same time when donor BM and spleen cells were intravenously injected. The recipients were monitored daily for survival and every days for body weight changes and clinical signs of GVHD. The clinical scoring was based on six parameters as used by others,^{10,14} including weight loss, posture, activity, fur texture, skin integrity, and diarrhea. A severity scale from 0-2 was used for each parameter, and the maximum score is 12.

Cell preparation: TCD-BM cells were collected by depleting CD4⁺, CD8⁺, and TCRβ⁺ cells from BM, using the magnetic purification system (Miltenyi Biotec). CD11⁺ DCs in lamina propria, liver, LN, and spleen were collected as previously described.^{20,33} Briefly, tissues were first digested using collagenase D (500 μg/ml and DNase I (50 U/ml) for about 30 min at 37° C. Then, CD11⁺ DCs were enriched with anti-CD11c magnetic beads (Miltenyi Biotec). CD103⁺ and CD103⁻CD11⁺ DC subsets (purity>99%) were isolated with flow cytometry sorting after magnetic enrichment of CD11c⁺ DCs. MNC cells from liver and gut were processed and collected as previously described. MNCs from skin were collected as follows: back skin (3×3 cm²) were cut into small pieces and digested in RPMI containing 10 mM HEES (Irvine Scientific), 0.01% DNase (Sigma), 0.27% collagenase (Sigma), and 1000 U of hyaluronidase at 37° C. for 1 hour. Skin MNCs were then isolated by Lymphocyte M.

Flow cytometric analysis: The following anti-mouse mAbs were purchased from BD Pharmingen, eBioscience, and R&D Systems: TCRβ (H57-597), CD4 (RM4-5), CD8α (53-6.7), B220 (RA3-6B2), CD11b/Mac-1 (M1/70), Gr-1(RB6-8C5), H-2^{q} (KH114), H-2^{b}(AF6-88.5), H-2^{d} (34-2-12), PD-1(CD279, J43), B7H1(CD274, 1-111A), CD11c (HL3), α4β7 (DATK32), CD103 (M290), CCR9 (CW-1.2), CCR4 (1G1), CCR10 (248918), CCR5 (HM-CCR5), CXCR3 (1C6/CXCR3), PDCA-1 (JF05-1C2.4.1), E-selectin/Fc Chimera, P-selectin/Fc chimera, and anti-IFN-γ(2E2). FACS was performed with a four-laser MoFlo Immunocytometry System (Dako Cytomation), and data were analyzed with FlowJo software (Tree Star), as described previously. The FoxP3 staining kit was purchased from eBioscience.

For intracellular staining, cells were stimulated with PMA/Inomycin in the presence of Golgi-Stop (BD Bioscience, SD) and cells were then harvested and stained for cytokines as previously described^{84,93}. Dead cells were excluded by Fixative Aqua Dead Cell Stain Kit (Invitrogen, CA).

Anti-CD3-based conditioning regimen and HCT: Anti-CD3 (145-2C11) was produced as previously described.⁹¹ WT or B7H1^{-/-} mice were injected i.v. with anti-CD3 at a dose of 5 μg/g body weight. Seven days after conditioning, mice were injected with BM (100×10⁶) and CD8⁺ T cells (20×10⁶) from C57BL/6 donors. The donor CD8⁺ T cells were injected again 5 days after injection. Unconditioned Rag2^{-/-} or B7H1^{-/-}Rag2^{-/-} mice were given one injection of donor CD8⁺ T cells (20×10⁶) and TCD-BM (2.5×10⁶) from C57BL/6 donors. CD8⁺ T cells from donor spleen were negatively selected by using a cocktail of biotin-conjugated antibodies against CD4, B220, DX5, CD11b, Ter-119, and anti-Biotin MicroBeads (Miltenuyi Biotec, Auburn, Calif.). The purity was >90%. In vivo BLI of BALB/c recipients transplanted with sorted CD8⁺ T cells from luc⁺ C57BL/6 donors and BM from nontransgenic C57BL/6 donors were performed as previously described,⁹¹ using an IVIS100 charge-coupled device imaging system (Xenogen).³³

Clinical GVHD scoring and histopathological analysis: Recipients were weighed before HCT and after every 3 to 4 days after HCT for up to 100 days. Recipients were also scored for clinical signs of GVHD including diarrhea, skin damage, and hunched posture as previously described.⁸⁴ Tissues were fixed in 10% phosphate-buffered formalin, embedded in paraffin, sectioned, and stained with hematoxylin and eosin (H&E) as previously described.⁴² Assessment of tissue damage was performed based on the previously described scoring system.^{36,40,84}

Hepatocyte isolation, RT-PCR and immunohistochemistry staining of B7H1: Isolation of mouse hepatocytes was performed according to the standard operation procedure previously described. Immunohistochemical staining was performed with formalin-fixed tissue sections as previously described.¹⁹

In vivo Bromodeoxyuridine (BrdU) labeling and in vitro T cell proliferation: HCT recipients were injected i.p. with BrdU (80 μg/g), twice over a 48 hour period, followed by tissue collection and analysis of T cell for BrdU incorporation using BrdU flow kit protocol (BD Pharmingen). For proliferation assay, sorted CD8⁺ T cells from recipients were isolated and stimulated with plated-bound anti-CD3 and soluble anti-CD28 with or without the addition of IL-2 (100 u/ml) for 72 hours. Proliferation was measured with [³H] incorporation as previously described.^{42,90,91}

B7H1^{-/-} bone marrow chimeric mice: B7H1^{-/-} Rag2^{-/-} mice were lethally irradiated (950 rads) and reconstituted with B7H1^{+/+-} Rag2^{-/-}BM cells (5×10⁶)(B7H1^{+/+} into B7H1^{-/-} chimeras) or B7H1^{-/-} Rag2^{-/-}BM cells (B7H1^{-/-} into B7H1^{+/+}). Reverse chimeric mice were also created by lethally irradiating B7H1^{+/+} Rag2^{-/-} mice and reconstituting them with either B7H1^{-/-} Rag2^{-/-}BM (B7H1^{-/-} into B7H1^{+/+} chimeras) or B7H1^{+/+} Rag2^{-/-}BM cells (B7H1^{+/+} into B7H1^{+/+} chimeras). BM cells (6.5×10⁶) from various donors were injected into each recipient mouse through tail vein 6 hours after irradiation. Depletion of circulating cells and reconstitution with donor cells was confirmed in all chimeric mice by evaluating B7H1 expression on blood and splenic CD11c⁺ DCs. Eight weeks after irradiation and BM reconstitution, chimeric mice were given 20×10⁶ CD8⁺ T cells plus 2.5×10⁶ TCD BM from C57BL/6. Mice were monitored for the development of GVHD.

Quantification of chemokine expression by real-time RT-PCR: Isolation of total tissue RNA and synthesis of first strand cDNA were described previously^{83,84}. mRNA was quantified by real-time quantitative PCR using Applied Biosystems 7300 Fast Real-Time PCR System (Applied Biosystems, Forest City, Calif.). The primers for chemokines were previously described in following publications: Ccl3-5,⁴⁹ Ccl17,³ Ccl22,³ Ccl25,⁶⁸ Ccl27,²⁸ Ccl28,²⁸ and Cxcl9-11.⁸⁴ Relative expression levels of genes were normalized within each sample to the house-keeping gene GAPDH and were presented relative to the expression in syngeneic transplantation recipients, in which irradiated BALB/c recipients were injected with 5×10⁶ syngenic TCD-BM cells as previously described.

MLR and in vitro inducing donor T expression of homing and chemokine receptors: Sorted CD4⁺, CD8⁺, or CD4⁺/CD8⁺ T cells (2×10⁵) from spleen of donor C57BL/6 mice were cultured with CD11c⁺ DCs (10⁵) from BABL/c host in a U-bottom 96-well plate for 4 days. The T cell homing and chemokine receptor expression was measured by flow cytometry and the T cell proliferation was measured by ³H-TdR incorporation, which were previously described.⁹¹

GVHD Histopathology and scoring: Colon, liver, and skin specimens were fixed in formalin before embedding in paraffin blocks. Tissue sections were stained with H&E.⁹¹ Assessment of tissue damage was performed based on scoring system.^{14,36} In brief, skin GVHD was scored on the basis of tissue damage in epidermis, dermis, and loss of subcutaneous fat; the maximum score is 10. Gut GVHD was scored on the basis of crypt apoptosis and lamina propria inflammation; the maximum score is 8. Liver GVHD was scored on the number of involved tracts and the severity of disease in each tract, and the maximum score is 8.

Statistical analysis: Comparison of survival groups was analyzed using the log-rank test with GraphPad Prism version 4.0 (GraphPad Software). Comparison of two means was analyzed using the unpaired two-tailed Student's t-test.

### Example 2

### Anti-CD3 Preconditioning Prevented GVHD but Retained GVL in TBI-Conditioned Recipients

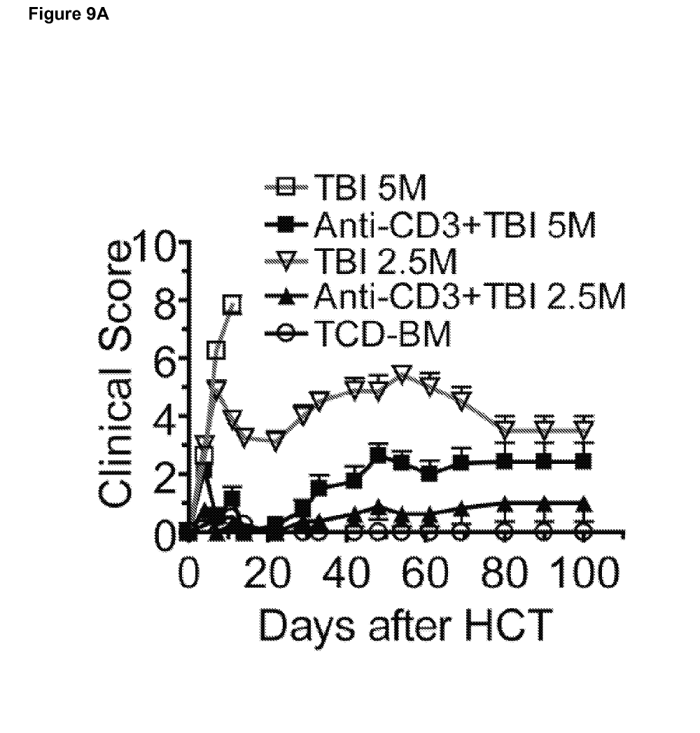

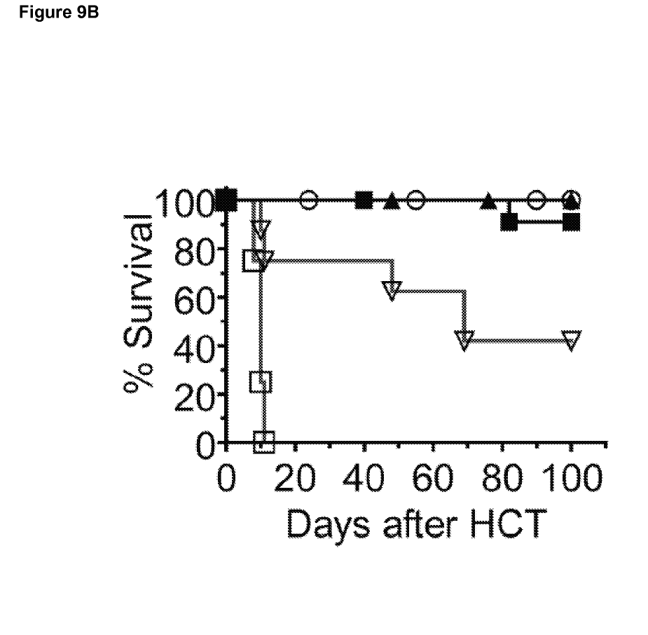

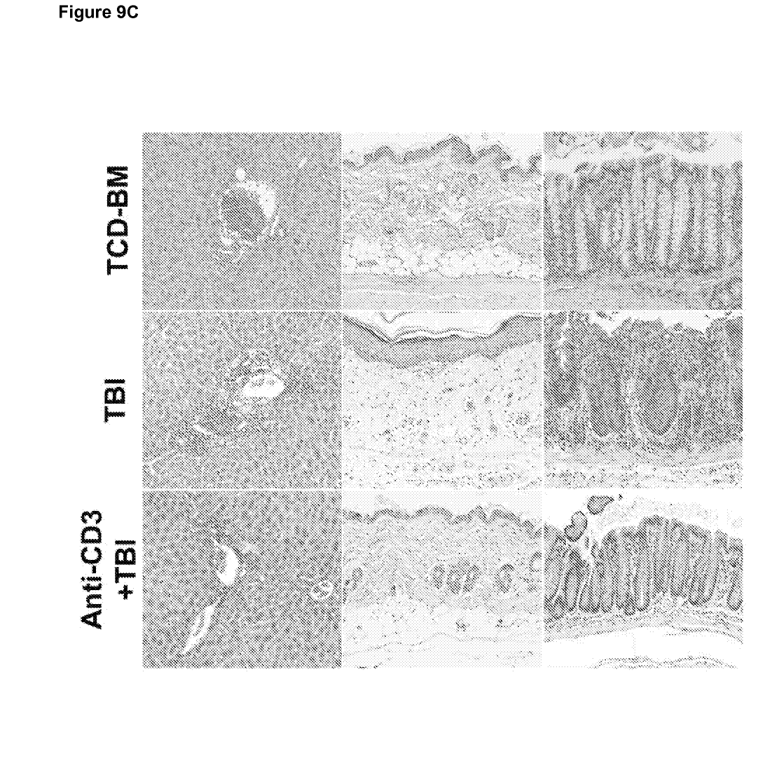

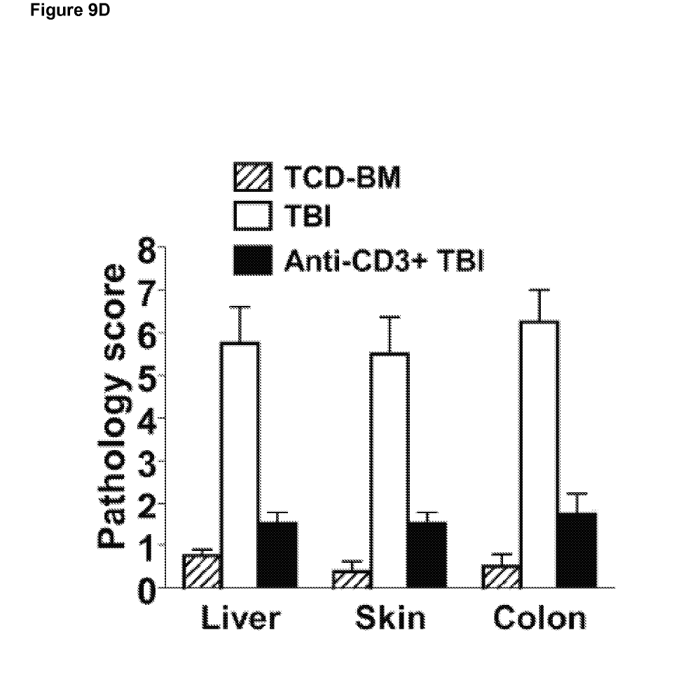

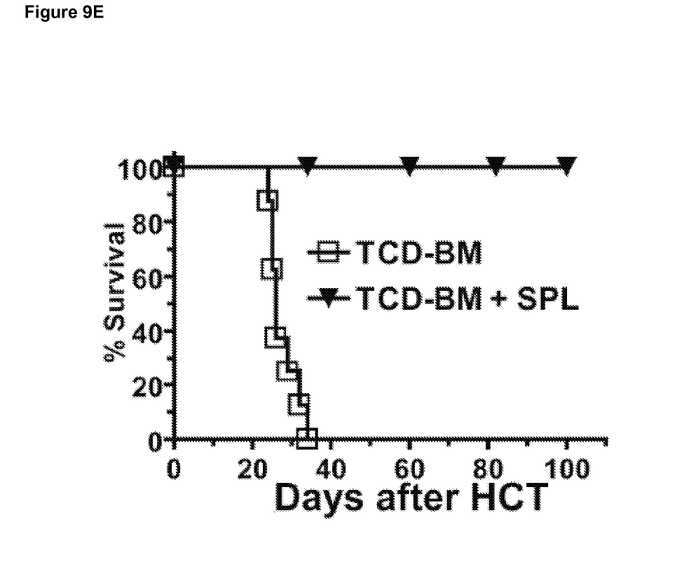

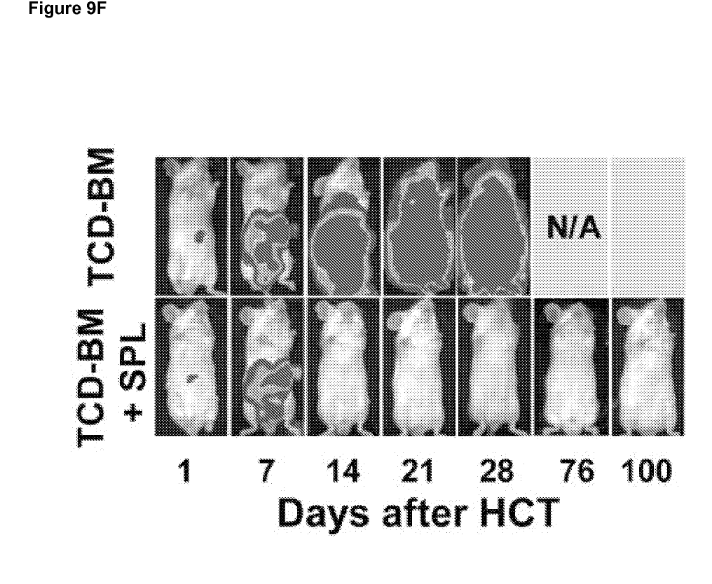

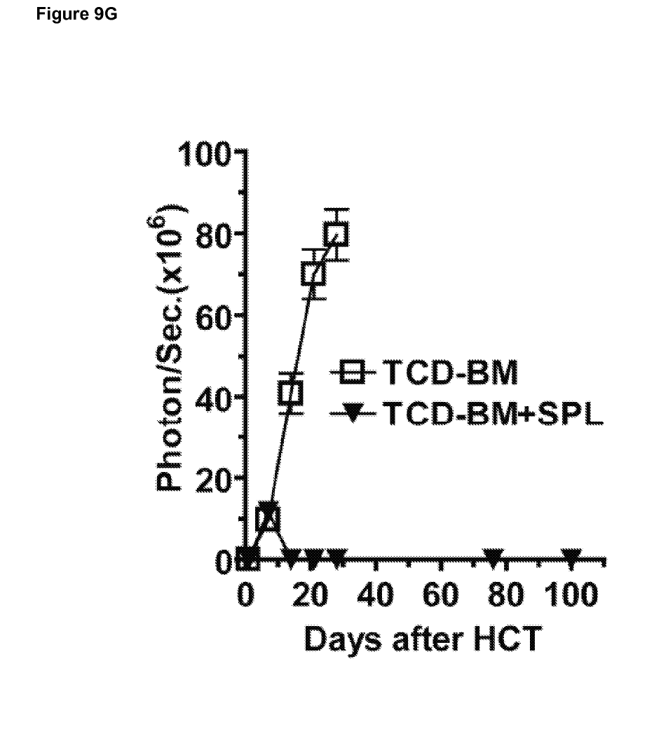

Anti-CD3 conditioning allowed donor CD8⁺ T cells to mediate GVL without causing clinical GVHD in non-irradiated recipients. To test whether anti-CD3 preconditioning could separate GVL from GVHD in TBI-conditioned recipients, BALB/c mice were injected with anti-CD3 (5 μg/g) or PBS (control) as preconditioning. Nine days after anti-CD3 injection, the mice were conditioned with TBI. At this time point, serum anti-CD3 was not detectable by blocking assay and the host TCRαβ⁺ cells were also not detectable in blood. Six hours after TBI, the recipients were injected I.V. with TCD-BM (5×10⁶) and spleen cells (2.5−5×10⁶) from C57BL/6 donor mice. The recipients were monitored daily for clinical GVHD including body weight, posture, diarrhea, and survival. While 5×10⁶ donor spleen cells induced severe clinical GVHD in control recipients without anti-CD3 preconditioning and all recipients died by 15 days after HCT, the same dose of donor cells induced only moderate clinical GVHD, and 91% ( 11/12) of the recipients survived for more than 100 days (P<0.01, FIGS. 9A and B). Similarly, 2.5×10⁶ donor spleen cells induced severe GVHD in the control recipients, and only 42% ( 5/12) of them survived for more than 100 days after HCT, in contrast, the same dose of donor cells induced minimum clinical GVHD in anti-CD3-preconditioned recipients and all the recipients survived for more than 100 days (FIGS. 9A and B). Therefore, anti-CD3 preconditioning markedly reduced clinical GVHD.

Additional experiments compared the histopathology of liver, skin, and colon of the recipients with or without anti-CD3 preconditioning 60 days after injection of 2.5×10⁶ donor spleen cells. It was observed that anti-CD3 preconditioning markedly reduced the infiltration and tissue damages in liver, skin, and colon (P<0.01, FIGS. 9C and D). Taken together, anti-CD3 preconditioning prevents induction of acute GVHD.

Whether anti-CD3 preconditioning could retain GVL while preventing GVHD was also verified using the following experiment: Luciferase transfected (Luc⁺) BCL1 leukemia/lymphoma cells (0.5×10⁶) were co-injected with donor TCD-BM (5×10⁶) and spleen cells (2.5×10⁶) into anti-CD3-preconditioned recipients 6 hours after TBI-conditioning. The control recipients were injected with TCD-BM and Luc⁺BCL1 cells only. The recipients were monitored for survival daily and were monitored for tumor growth using in vivo BLI weekly. Luc⁺ BCL1 tumor cells grew rapidly in recipients given TCD-BM without donor spleen cells, and killed the recipients 30-40 days after HCT (FIG. 9 E-G). In contrast, after a transient growth, Luc⁺ BCL1 tumor cells were eliminated in the recipients transplanted with both TCD-BM and spleen cells, and all the recipients survived for more than 100 days with little clinical GVHD (P<0.01, FIG. 9 E-G). These results further demonstrate that anti-CD3 preconditioning prevents GVHD but retains GVL activity.

### Example 3

### Anti-CD3 Preconditioning Reduced Donor Th1 Differentiation and Donor T Infiltration of GVHD Target Tissues

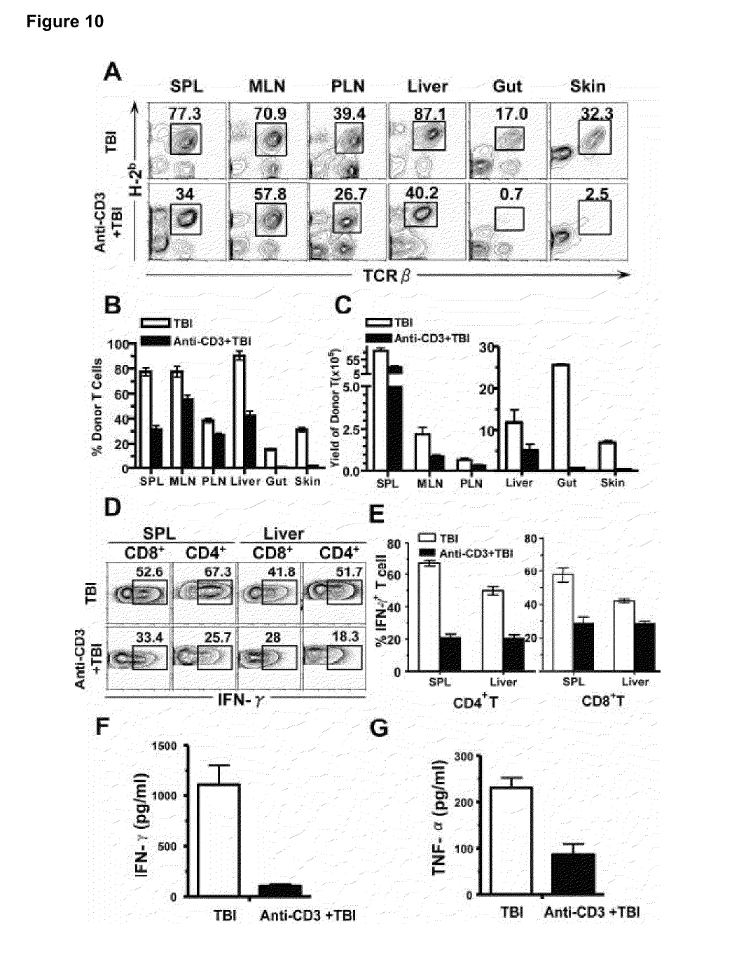

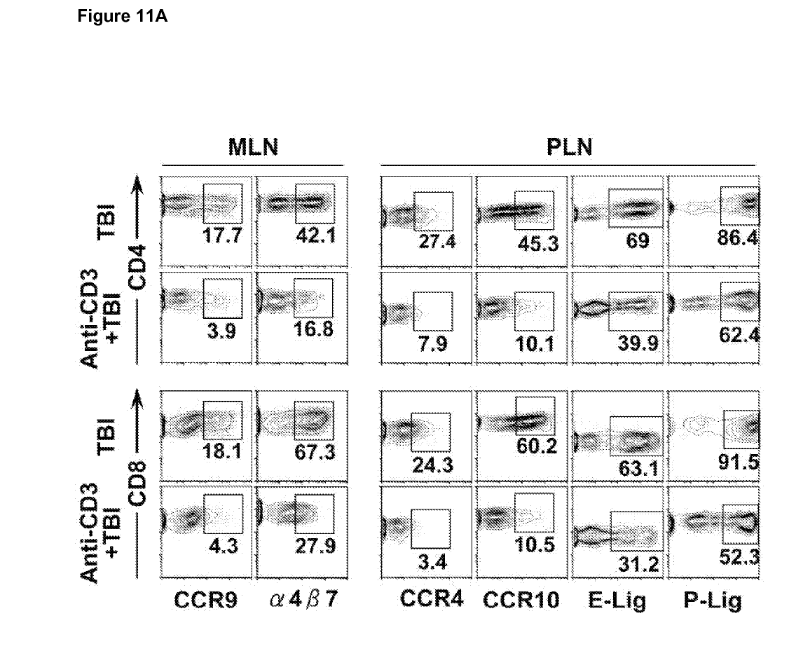

Donor T cell expansion in TBI-conditioned recipients reached first peak 5 days after HCT. The percentage and yield of donor T cells in lymphoid and GVHD target tissues (liver, gut, and skin) in the recipients with or without anti-CD3 preconditioning, 5 days after injection of donor TCD-BM and spleen cells (5×10⁶) was compared. It was found that the percentage and the yield of donor T cells in the spleen, MLN, and PLN of the recipients with anti-CD3 preconditioning were about 2-fold lower than that of the control recipients, although this difference was significant (P<0.01, FIG. 10A-C). In contrast, the percentage and yield of donor T cells in skin and gut of the recipients preconditioned with anti-CD3 was more than 15 fold lower than that of the control recipients (P<0.01, FIG. 10A-C) and was similar to results for spleen. These results demonstrate that anti-CD3 preconditioning markedly inhibits donor T cell migration into GVHD target tissues such as gut and skin in TBI-conditioned recipients.

Because IFN-γ-producing Th1 and Tc1 cells may play an important role in mediating acute GVHD target tissue damage, the percentage of the IFN-γ⁺ donor CD4⁺ was compared with CD8⁺ T cells in spleen and liver of the recipients. Anti-CD3 preconditioning reduced the percentage of IFN-γ⁺ cells among total CD4⁺ and CD8⁺ T cells by about two-fold in the spleen and liver as compared to that of the control recipients (P<0.01, FIGS. 10D & E). Consistently, anti-CD3 preconditioning reduced the serum levels of Th1 cytokine IFN-γ and TNF-α by 2-5 fold (P<0.01, FIGS. 10F & G). These results demonstrate that anti-CD3 preconditioning leads to significant reduction of donor Th1 differentiation.

### Example 4

### Anti-CD3 Preconditioning Inhibited Upregulation of Chemokine Receptors by Donor T Cells as Well as Chemokine Release by GVHD Target Tissues In TBI-Conditioned Recipients

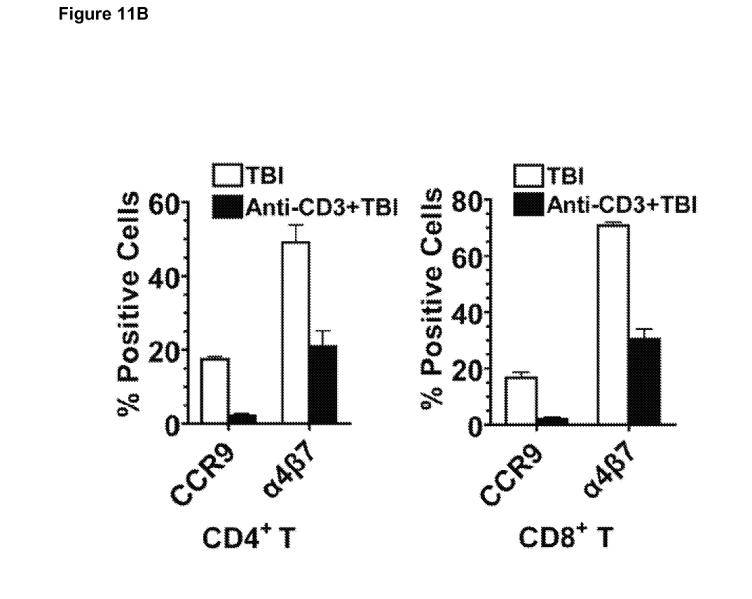

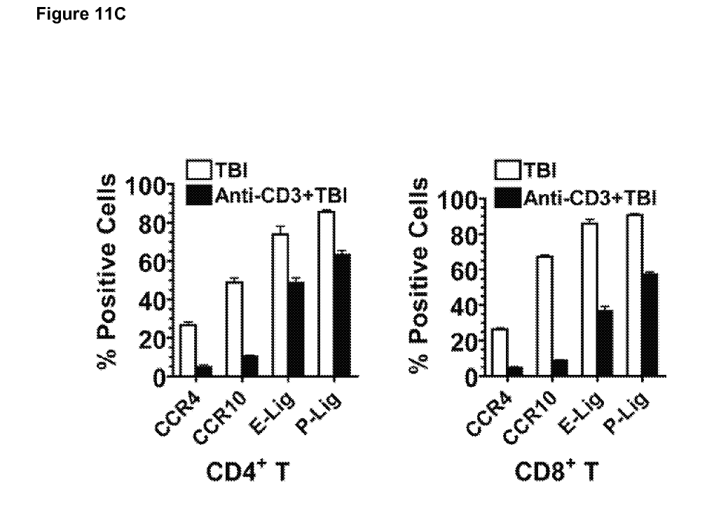

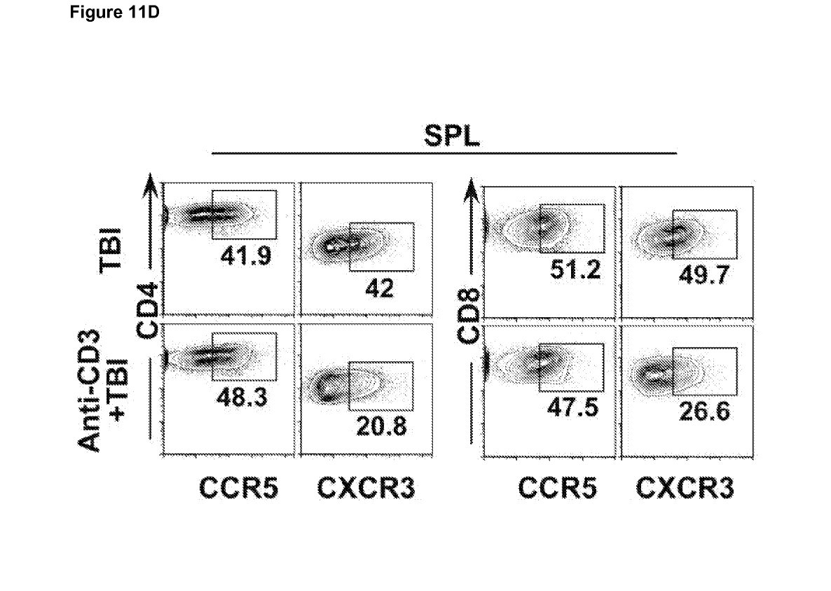

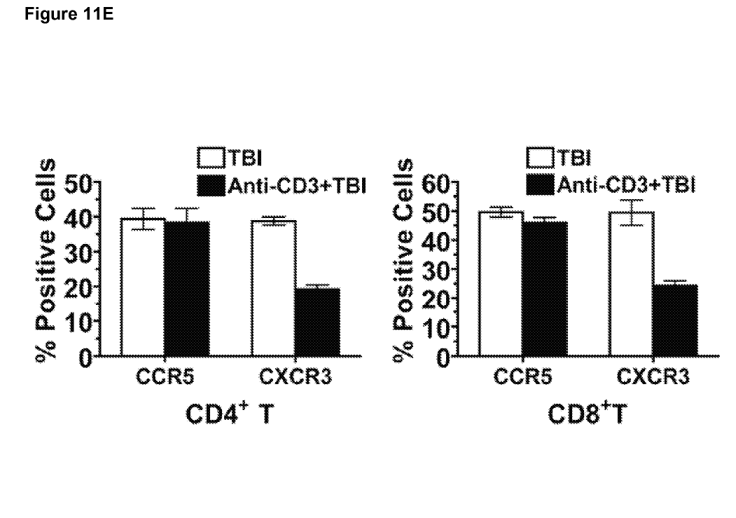

Homing and chemokine receptors expressed by donor T cells as well as chemokines released by GVHD target tissues play a role in donor T cell migration into GVHD target tissues. Donor T expression of homing was compared with chemokine receptors in MLN and PLN of recipients with or without anti-CD3 preconditioning. Anti-CD3 preconditioning reduced the percentage of α4β7⁺ CD4⁺ and CD8⁺ T in MLN by more than 2-fold and reduced the percentage of CCR9⁺ CD4⁺ and CD8⁺ T cells by more than 4-fold (P<0.01, FIGS. 11A and B). Similarly, anti-CD3 preconditioning reduced the percentage of E-Lig⁺ or P-Lig⁺ CD4⁺ and CD8⁺ T cells in PLN by about 2-fold and reduced the percentage of CCR4⁺ or CCR10⁺ CD4⁺ and CD8⁺ T cells by about 5 fold (P<0.01, FIGS. 11A & B). These results demonstrate that anti-CD3 preconditioning inhibit donor T cell upregulation of homing and chemokine receptors in host draining LNs.

In addition, donor T expression of CCR5 and CXCR3 were compared in spleen of recipients with or without anti-CD3 preconditioning. Although there was no significant difference in the percentage of donor CCR5+CD4⁺ and CD8⁺ T cells, there was a 2-fold reduction in the percentage of CXCR3⁺ CD4⁺ and CD8⁺ T cells in the anti-CD3-preconditioned recipients (FIGS. 11D & E). These results demonstrate that anti-CD3 preconditioning can also inhibit donor T expression of some non-tissue specific chemokine receptors.

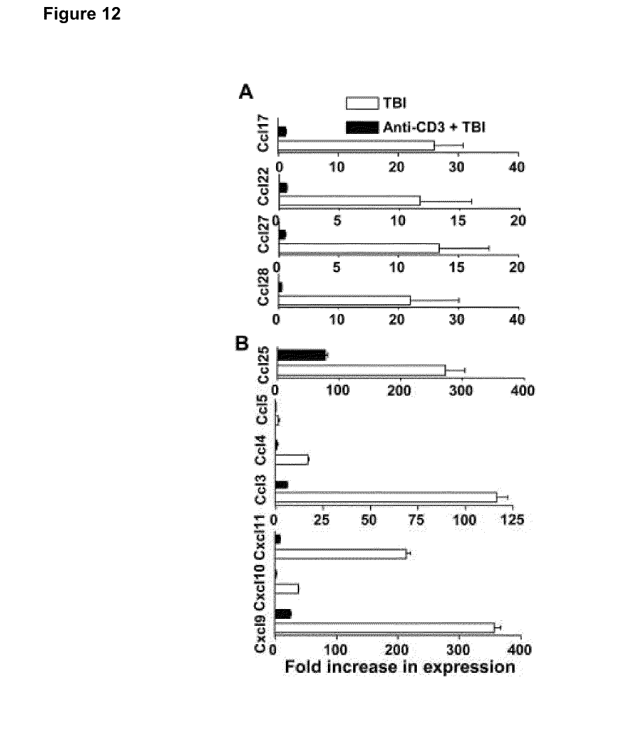

Next, the GVHD target tissue expression of chemokines, including skin tissue expression of CCL17 was compared with CCL22 (CCR4 ligand) and CCL27 and CCL28 (CCR10 ligand), the gut tissue expression of CCL25 (CCR9 ligand), and non-tissue specific chemokines CCL3-5 (CCR5 ligand) and CXCL9-11 (CXCR3 ligand). Anti-CD3 preconditioning reduced the skin tissue expression of CCL17, CCL22, CCL27, and CCL28 by more than 10-fold and reduced gut tissue expression of CCL25 by more than 3-fold fold (P<0.01, FIG. 12). In addition, anti-CD3 preconditioning reduced the gut, skin, and liver tissue expression of CCL3-5 and CXCL9-11 by more than 10 fold (P<0.01, FIG. 12B). These results demonstrate that anti-CD3 preconditioning inhibits the release of inflammatory chemokines in GVHD target tissues trigged by TBI-conditioning.

### Example 5

### Anti-CD3 Preconditioning Reduced CD103⁺ DCs in MLN and Down-Regulated MLN DC's Capacity in Imprinting Donor T Cell Expression of Gut Homing and Chemokine Receptors

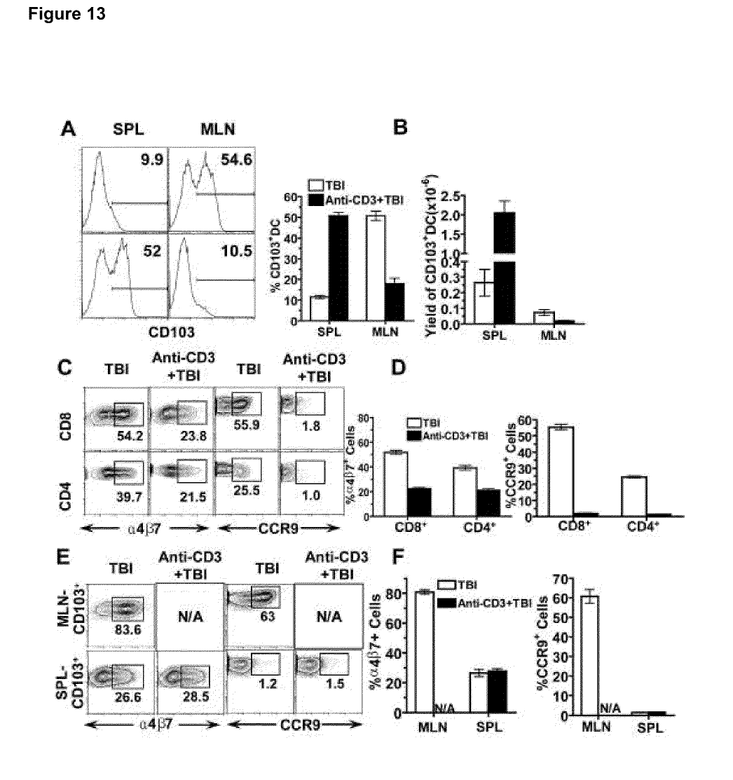

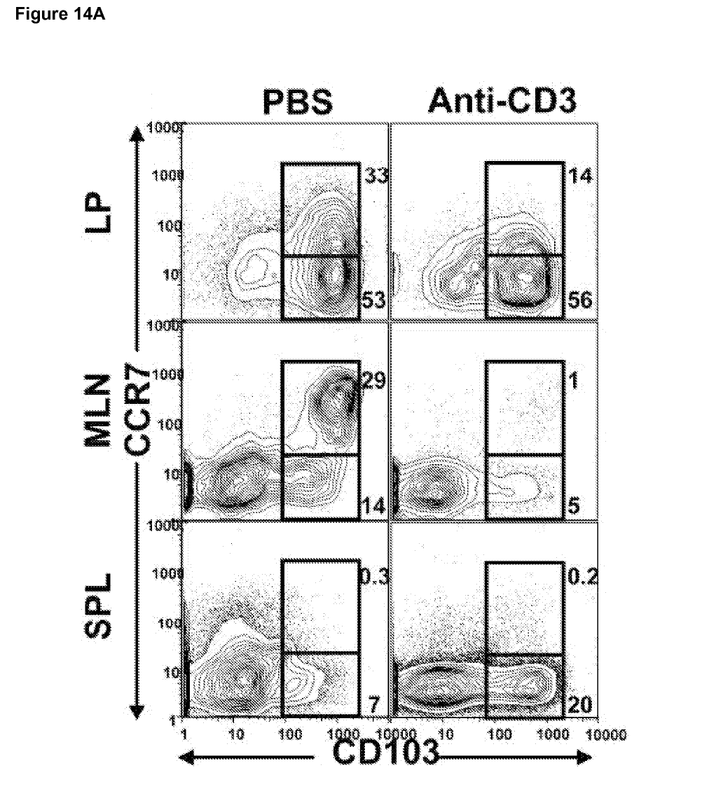

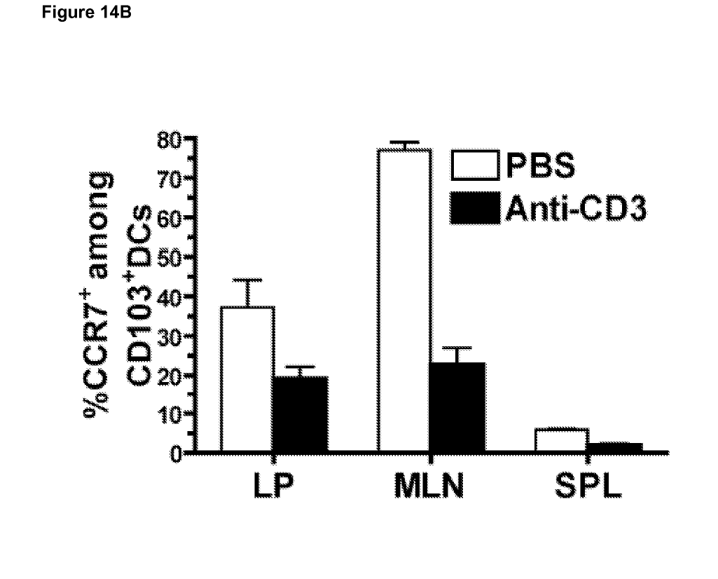

A marked reduction of α4β7⁺ CCR9⁺ donor T cells in MLN of recipients preconditioned with anti-CD3 was observed (FIG. 11), and it was reported that CD103⁺ DCs in MLN induced T cell expression of α4β7 and CCR9.³³ The effect of anti-CD3 preconditioning on the percentage and yield of CD103⁺ DCs in MLN was evaluated as well as the capacity of MLN DCs in inducing donor T expression of gut homing α4β7 and CCR9 chemokine receptors. Anti-CD3 preconditioning reduced the percentage and yield of CD103⁺ DCs in MLN by about 5-fold, but increased the percentage and yield of CD103⁺ DCs in spleen by more than 5-fold (P<0.01, FIGS. 13A & B). These results demonstrate that anti-CD3 preconditioning reduces CD103⁺ DCs in MLN.

Next, the MLN DC's capacity in inducing donor T expression of α4β7 was compared with CCR9 in an in vitro culture. CD11c⁺ DCs from MLN of BALB/c mice without anti-CD3 preconditioning induced about 50% of donor CD8⁺ T cells to express α4β7 and CCR9 and induced 40% and 25% of donor CD4⁺ T cells to express α4β7 and CCR9, respectively. In contrast, anti-CD3 preconditioning reduced the DC's capacity in inducing donor CD4⁺ and CD8⁺ T expression of α4β7 by two-fold and almost completely abrogated the DC's capacity in inducing donor CD4⁺ and CD8⁺ T expression of CCR9 (P<0.01, FIGS. 13 C and D). These results demonstrate that marked reduction of CD103⁺ DCs in MLN after anti-CD3 preconditioning leads to marked reduction of MLN DC's capacity in induction of donor T expression of α4β7 and CCR9 receptors.

Because anti-CD3 preconditioning markedly increased the percentage and yield of CD103⁺ DCs in spleen (FIGS. 13 A and B), the spleen was compared with MLN CD103⁺ DCs in inducing donor T expression of α4β7 and CCR9. Accordingly, CD103⁺ DCs were sorted from MLN and spleen of control mice or anti-CD3-preconditioned mice. The sorted CD103⁺ DCs were co-cultured with sorted donor CD8⁺ T cells. CD103⁺ DCs from MLN of the control mice without anti-CD3 preconditioning induced more than 80% or 60% of donor CD8⁺ T cells to express α4β7 or CCR9, respectively. In contrast, CD103⁺CD11c⁺ DCs from the spleen of the same mice induced 3-fold less α4β7⁺ and 50-fold less CCR9⁺ donor CD8⁺ T cells (P<0.01, FIGS. 13 E and F). Similarly, CD103⁺ DCs from spleen of anti-CD3 preconditioned mice failed to induce donor CD8⁺ T expression of CCR9 (FIGS. 13 E & F). These results show that CD103⁺ DCs in MLN but not in spleen can efficiently induce donor T cells to upregulate both α4β7 and CCR9. Although MLN DCs induce CCR9⁺FoxP3⁺ Treg cells when co-cultured with OVA specific transgenic CD4⁺ T cells, host MLN DCs induce alloreactive donor CD4⁺ and CD8⁺ T cells to express only CCR9 but not FoxP3.

### Example 6

### Reduction of CD103⁺ DCs in MLN by Anti-CD3 Preconditioning was Associated with Downregulation of CCR7 on CD103⁺ DCs

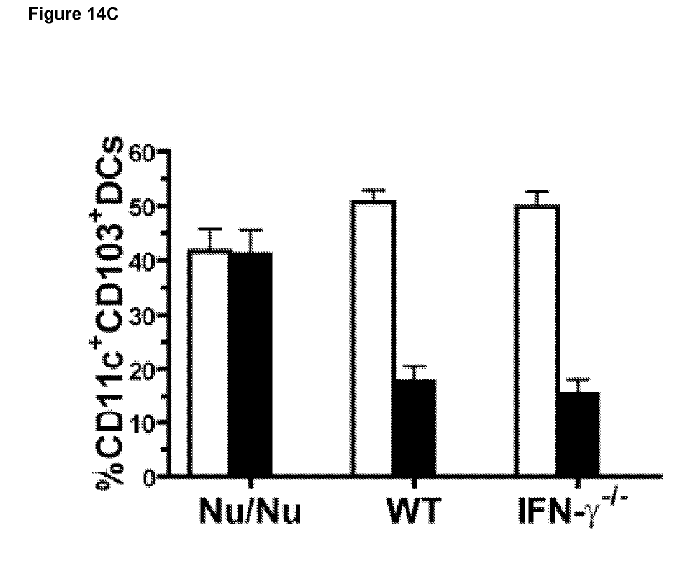

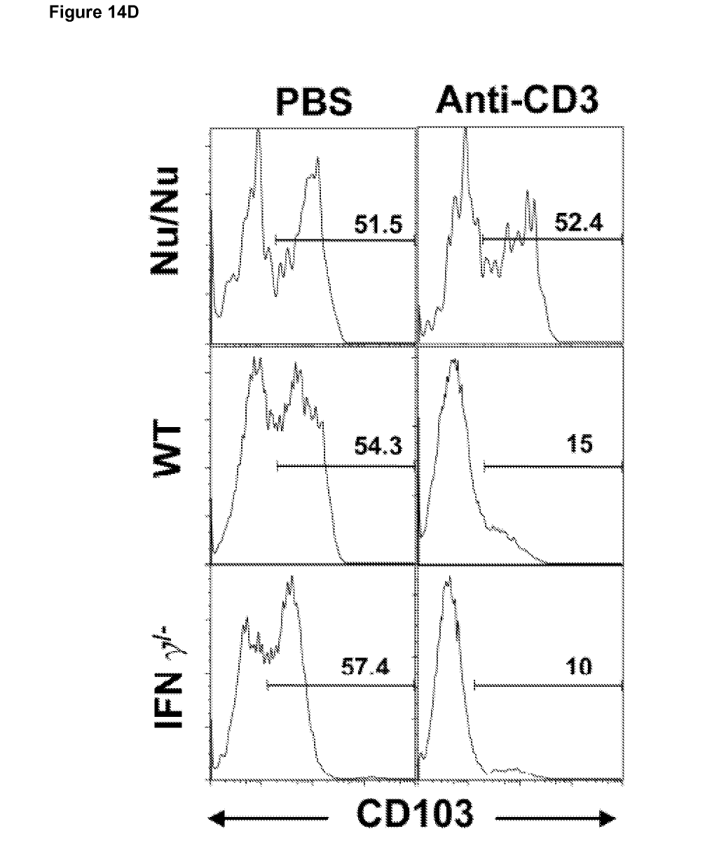

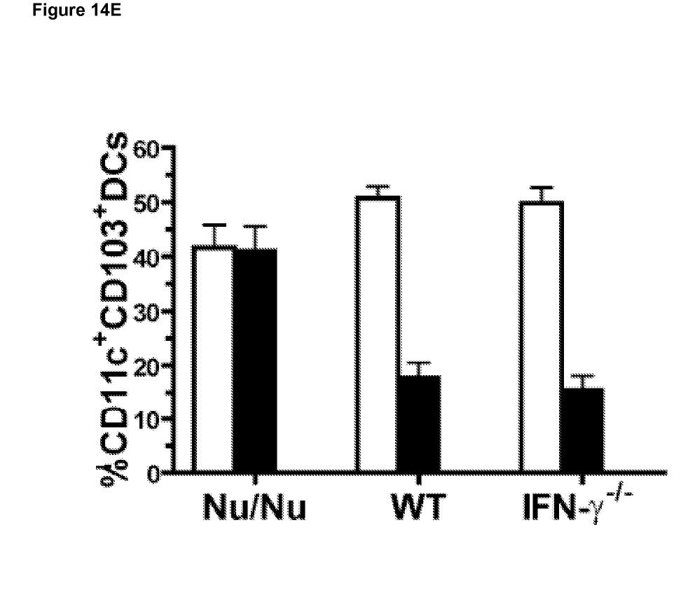

To test whether CD103⁺ DCs migration from lamina propria (LP) to MLN was dependent on their expression of CCR7, CCR7 expression by CD103⁺ DCs in LP and MLN with or without anti-CD3 preconditioning were compared. After anti-CD3 preconditioning, most (>85%) of the CD11c⁺ DCs from LP were CD103⁺. The percentage of CCR7⁺CD103⁺ DCs and their CCR7 expression levels in LP were reduced by more than 2-fold (P<0.01, FIG. 14 A-C); accordingly, the percentage of CCR7⁺CD103⁺ DCs among total CD11c⁺ DCs or among residual CD103⁺ DCs in MLN was reduced by about 3-fold or 4-fold, respectively, and their CCR7 expression levels was reduced by 5-fold (P<0.01, FIG. 14 A-C). There was no increase of CCR7⁺CD103⁺ DCs in spleen, although the CCR7-CD103⁺ DCs were increased by 4-fold (P<0.01, FIG. 14 A-C). These results demonstrate that CD103⁺ DCs with high-level expression of CCR7 are enriched in MLN and that that reduction of CD103⁺ DCs in MLN after anti-CD3 preconditioning is associated with downregulation of CCR7 expression by CD103⁺ DCs in LP. This also demonstrates that anti-CD3 preconditioning may prevent CD 103⁺ DC migration from LP to MLN.

### Example 7

### Reduction of CD103⁺ DCs in MLN by Anti-CD3 Preconditioning Required Activation of Host T Cells

To test whether anti-CD3 activation of host T cells was necessary for reduction of CD103⁺ DCs in MLN, the percentage of CD103⁺ DCs in MLN of wild-type BALB/c and T cell-deficient BALB/c nu/nu mice were compared before and after anti-CD3 preconditioning. While anti-CD3 preconditioning always markedly reduced the percentage of CD103⁺ DCs in MLN of wild-type mice, it resulted in little change in BALB/c nu/nu mice (FIGS. 14 D and E). These results demonstrate that anti-CD3 activation of host T cells is required for reduction of CD103⁺ DCs in MLN.

Because reduction of CD103⁺ DCs in MLN after anti-CD3 preconditioning was associated with down-regulation of CCR7 expression by the CD103⁺ DCs (FIG. 14 A-C), and anti-CD3 preconditioning led to an elevation of serum IFN-γ, a cytokine that regulates chemokine receptor expression, CD103⁺ DC percentage in MLN of IFN-γ^{-/-} mice with or without anti-CD3 preconditioning. Anti-CD3 preconditioning still markedly reduced the percentage of CD103⁺ DCs in MLN of IFN-γ^{-/-} mice (P<0.01, FIGS. 14 D & E). These results demonstrate that IFN-γ is not required for reduction of CD103⁺ DCs in MLN by anti-CD3 preconditioning.

### Example 8

### Donor CD8⁺ T Cells Induced GVHD in B7H1^{-/-} but not Wild Type Recipients Conditioned with Anti-CD3

Donor CD8⁺ T cells augmented donor hematopoietic cell engraftment and mediated graft versus leukemia (GVL) activity without GVHD in MHC-mismatched anti-CD3 conditioned recipients. Anti-CD3-conditioning led to not only markedly reduction of donor CD8⁺ T cell infiltration of GVHD target tissues but also anergy or unresponsive of the infiltrating CD8⁺ T cells in GVHD target tissues such as liver. B7H1, a co-inhibitory molecule, is constitutively expressed by hematopoietic cells such as antigen-presenting cells (APCs), and its expression in tissue parenchymal cells is induced by IFN-γ. B7H1 interaction with its ligand PD-1 on activated T cells leads to anergy and apoptosis of the T cells. Therefore, whether tissue cell expression of B7H1 played a role in GVHD prevention was tested in the recipients conditioned with this radiation-free anti-CD3-based regimen.

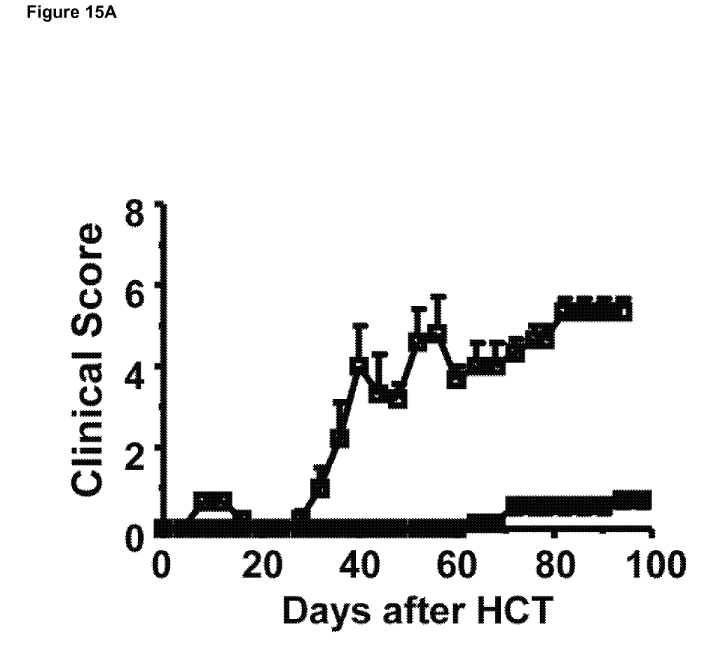

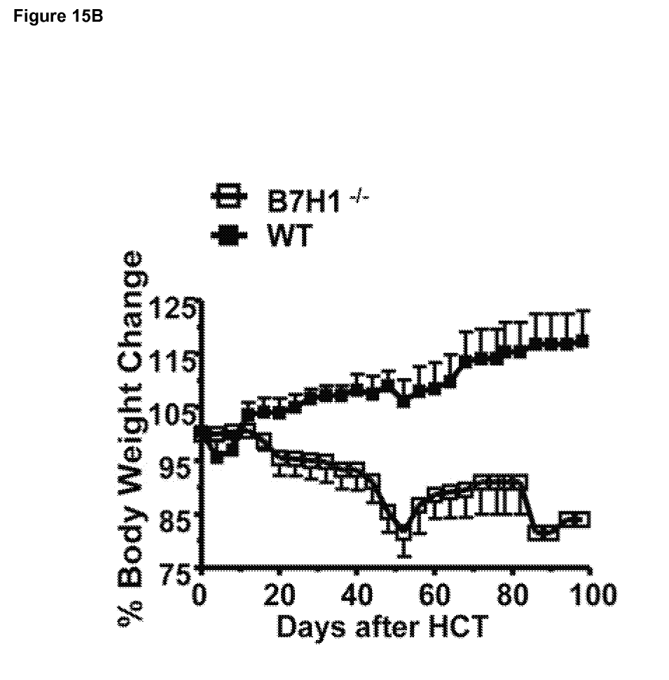

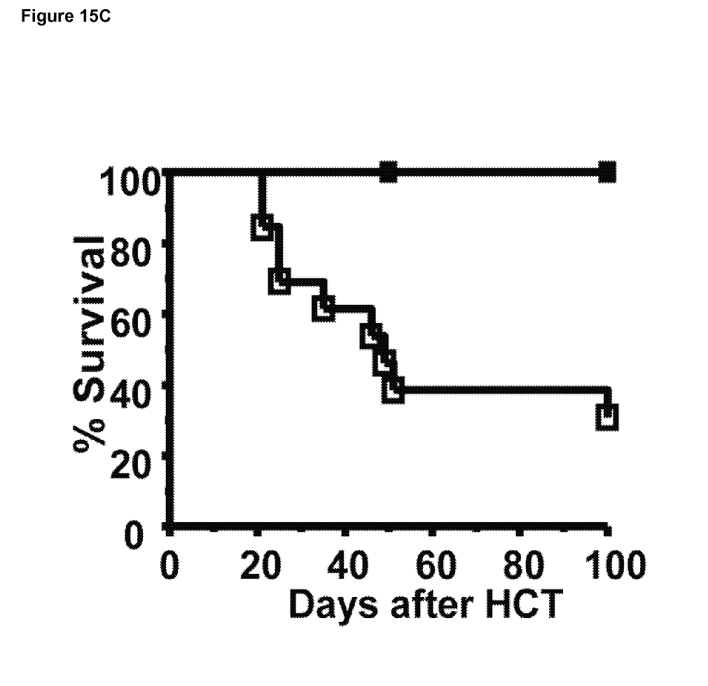

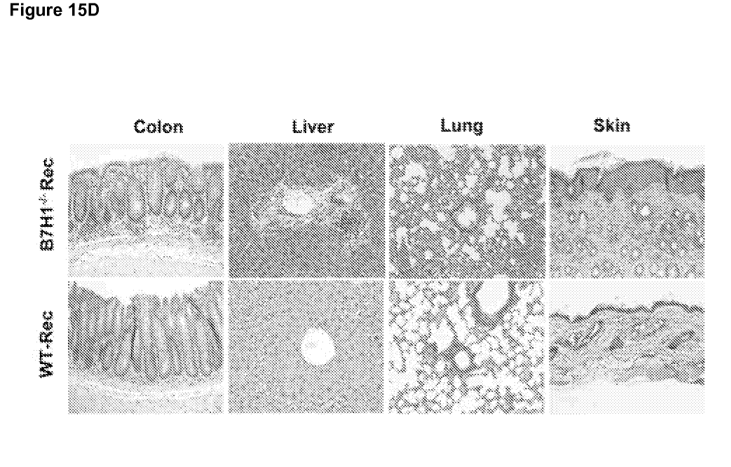

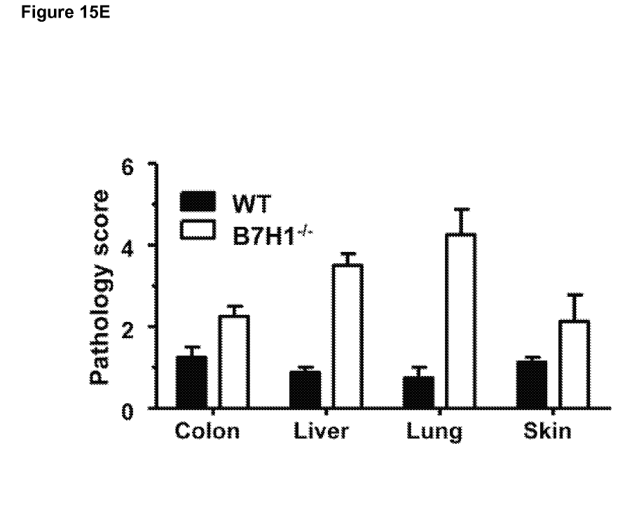

Accordingly, donor CD8⁺ T cells (20×10⁶) and BM cells (100×10⁶) were transplanted into anti-CD3-conditioned wild-type (WT) or B7H1^{-/-} recipients, and the CD8⁺ T cells were injected again 5 days after first injection. All the recipients developed complete chimerism. While the WT recipients showed little signs of GVHD and all of them survived for more than 100 days, the B7H1^{-/-} recipients showed severe signs of clinical GVHD including diarrhea, hunched back, raffled fur, and weight loss, and 75% of them died within 60 days after HCT (P<0.01, FIG. 15 A-C). Donor BM cells alone did not induce chimerism.

Furthermore, histopathology of GVHD target tissues of the residual B7H1^{-/-} recipients 100 days after HCT showed that, compared to the WT recipients, the B7H1^{-/-} recipients had much more severe infiltration and tissue damage in colon, liver, lung and skin (P<0.01, FIGS. 15 D & E). These results demonstrate that host tissue cell expression of B7H1 plays an important role in GVHD prevention in anti-CD3-conditioned recipients.

### Example 9

### Allogeneic HCT Led to Up-Regulation of B7H1 Expression in GVHD Target Tissues

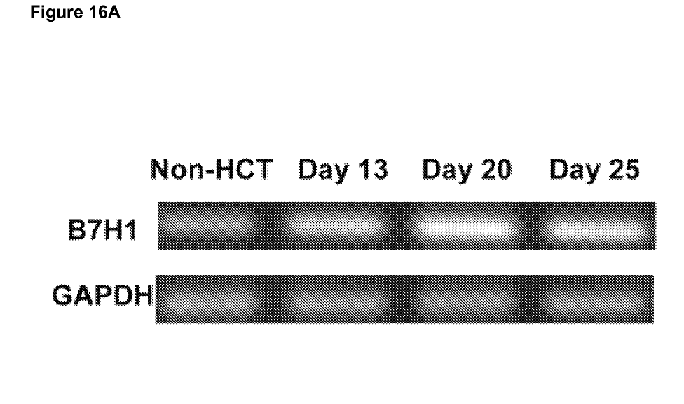

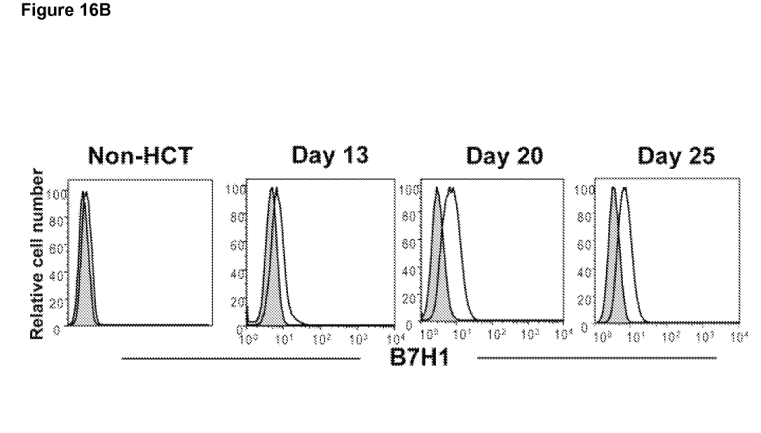

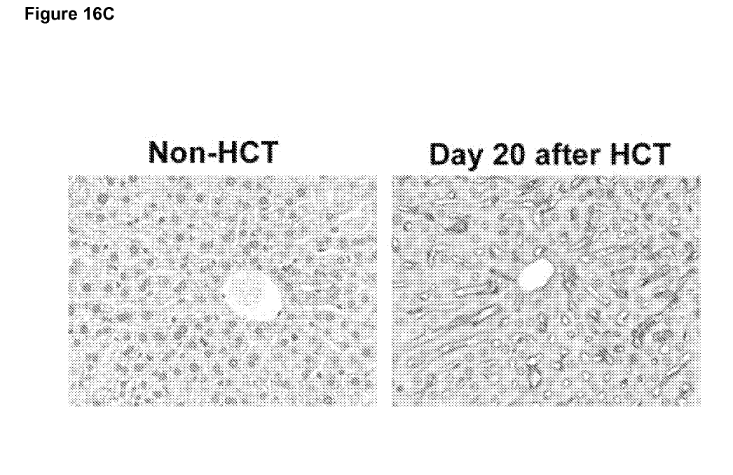

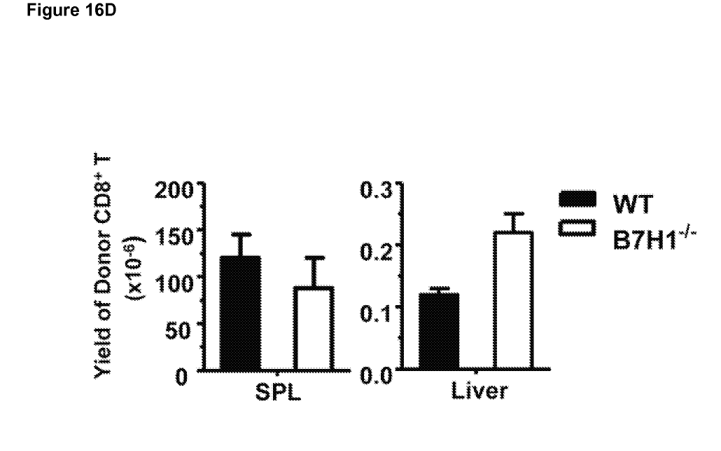

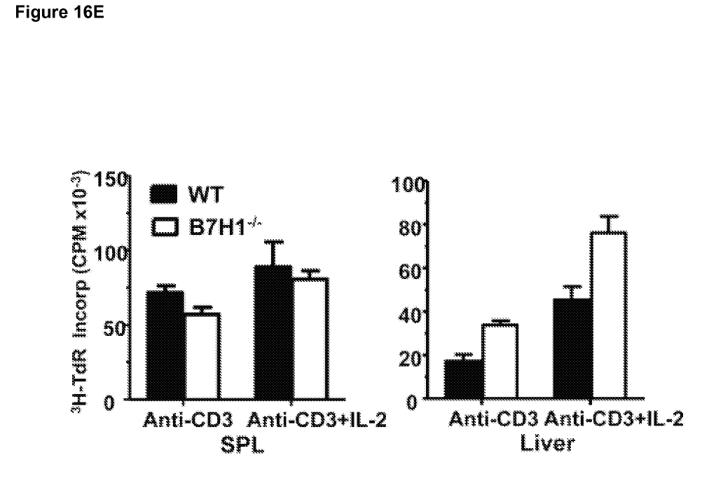

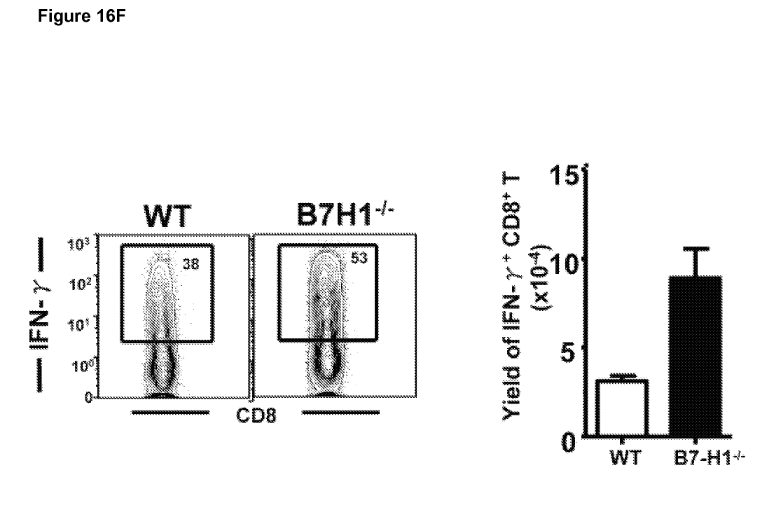

Next, the tissue parenchymal cell expression of B7H1 was measured before and after HCT, using RT-PCR, flow-cytometry, and immunohistochemistry. Taking hepatocytes as an example, it was found that hepatocyte expression of B7H1 mRNA was at low level before HCT, and it was gradually increased after HCT and reached the peak by 20 days after HCT (FIG. 16A). Consistently, hepatocyte expression of surface B7H1 was not detectable before HCT, and the expression was induced by HCT and reached to a peak by 20 days after HCT (FIG. 16B). The expression of surface B7H1 by hepatocytes was confirmed by immunohistochemical staining of B7H1 on hepatocytes before and 20 days after HCT (FIG. 16C). Similarly, the B7H1 expression on colon epithelial cells was also up-regulated after HCT as measured by RT-PCR or immunohistochemistry. Taken together, GVHD target tissue parenchymal cells were induced to express B7H1 after HCT.

### Example 10

### Host Tissue Expression of B7H1 was Associated with Reduction of Infiltration and Induction of Donor T Cell Anergy in Anti-CD3-Conditioned Allogeneic Recipients

Next, the yield and proliferation capacity of donor CD8⁺ T cells were compared in spleen and liver of WT and B7H1^{-/-} recipients, 20 days after HCT. Although the yield of donor T cells in spleen was not significantly different (P>0.5, FIG. 16D, left panel), the yield of donor CD8⁺ T cells in liver of B7H1^{-/-} was 2-fold higher than WT recipients (P<0.01, FIG. 16D, right panel). Furthermore, although the proliferation of donor T cells from spleen in response to anti-CD3/CD28 stimulation was similar (P>0.5, FIG. 16E, left panel), the proliferation of donor T cells from liver of B7H1^{-/-} recipients was 2-fold higher than WT recipients (P<0.01). In addition, the proliferation of donor CD8⁺ T cells from the liver of both WT and B7H1^{-/-} recipients was augmented about 2-fold by addition of IL-2 to the culture (P<0.01, FIG. 16E, right panel). Furthermore, the percentage of IFN-γ⁺CD8⁺ T cells in the liver of B7H1^{-/-} recipients was 2-fold higher compared to WT recipients (P<0.01, FIG. 16F). These results demonstrate that tissue expression of B7H1 is associated with anergy of infiltrating alloreactive donor T cells.

### Example 11

### Host Tissue Expression of B7H1 was Associated with GVHD prevention in unconditioned Rag2^{-/-} recipients

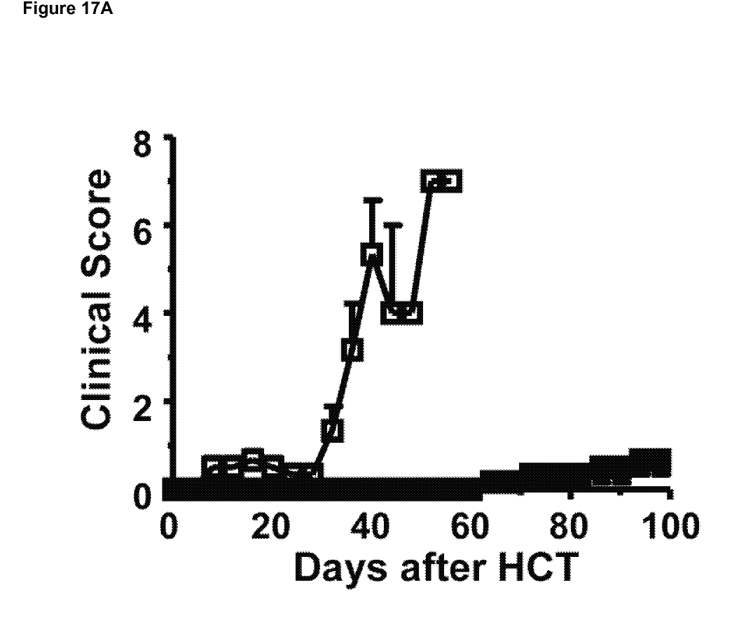

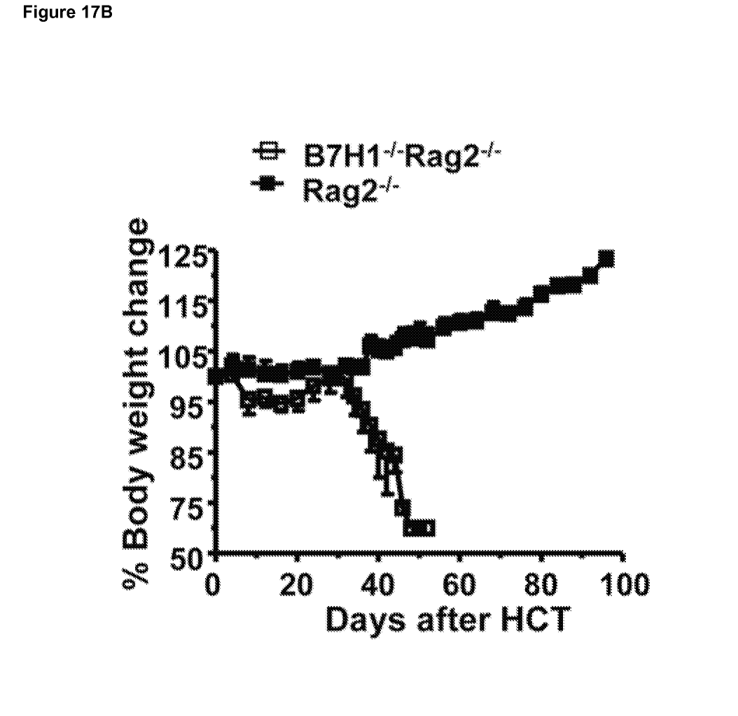

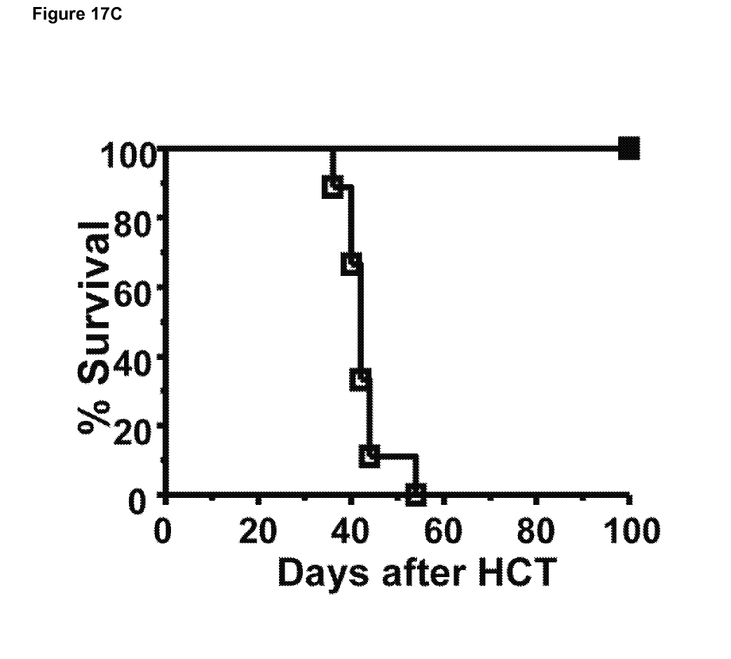

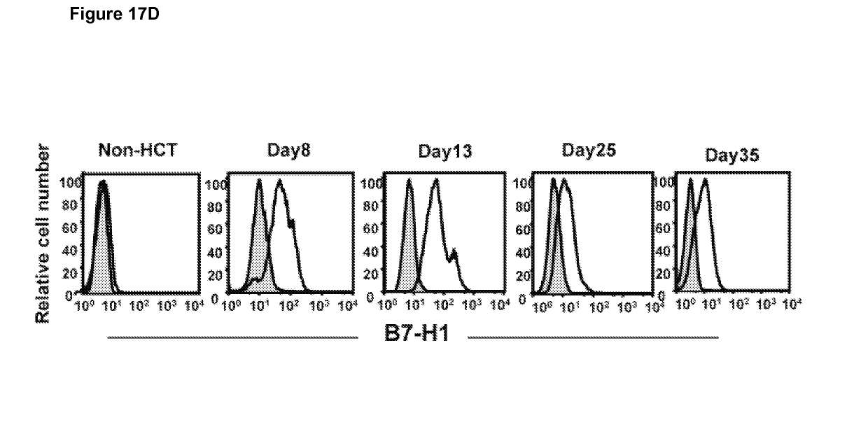

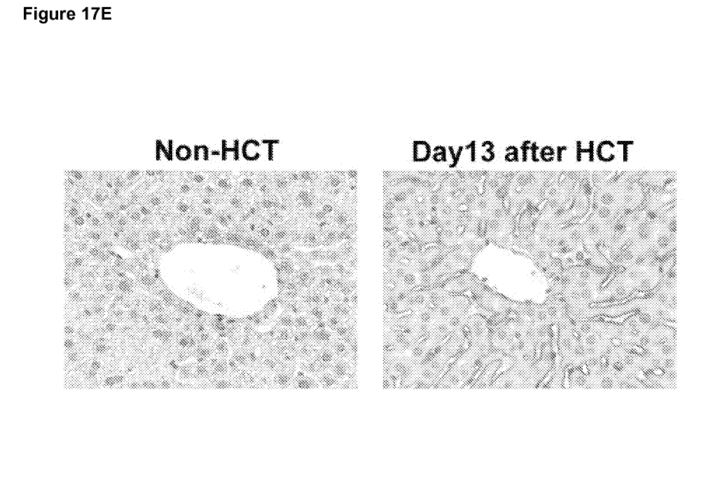

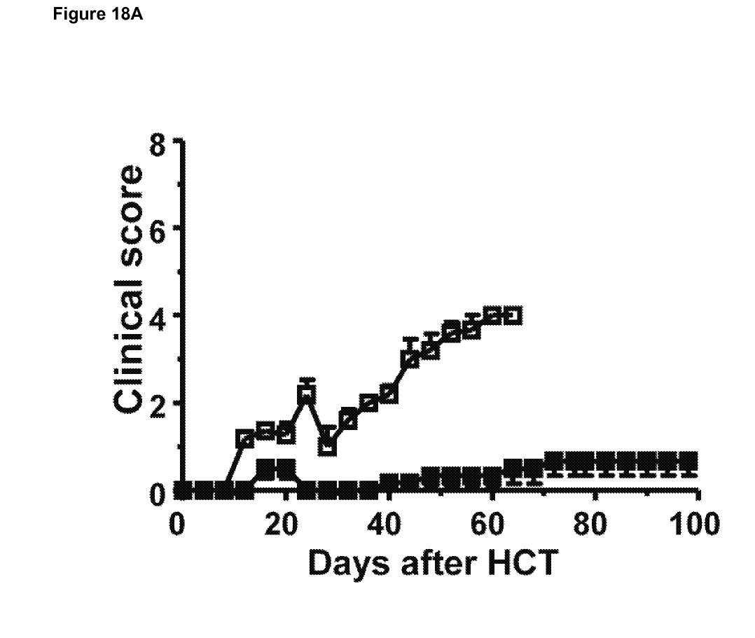

To further test the role of tissue expression of B7H1 in GVHD prevention and exclude the possibility that residual anti-CD3 played a critical role in GVHD prevention in anti-CD3-conditoned recipients, the role of tissue expression of B7H1 in GVHD prevention was tested in unconditioned Rag2^{-/-} recipients. Rag2^{-/-} recipients are deficient in T and B cells and can not reject donor T cells. Accordingly, sorted CD8⁺ T cells (20×10⁶) and TCD-BM cells (2.5×10⁶) from C57BL/6 donors were transplanted into B7H1 sufficient Rag2^{-/-} or B7H1 deficient B7H1^{-/-}Rag2^{-/-} recipients. While Rag2^{-/-} recipients developed little signs of GVHD and all survived for more than 100 days after HCT, the B7H1^{-/-}Rag2^{-/-} recipients developed severe clinical GVHD with diarrhea and weight-loss and all died within 60 days after HCT (P<0.01, FIG. 17A-C). TCD-BM cells alone did not induce chimerism. Furthermore, as compared to Rag2^{-/-} recipients, the B7H1^{-/-}Rag2^{-/-} recipients showed much more severe infiltration and tissue damage in colon, liver, lung and skin (P<0.01, FIG. 17).

Next, the tissue expression of B7H1 was measured before and after HCT as described above. Consistent with anti-CD3-conditioned WT BALB/c recipients, the hepatocyte surface expression of B7H1 was not detectable before HCT, and it was gradually increased after HCT and reached a peak 13 days after HCT (FIG. 17D). The hepatocyte surface expression of B7H1 after HCT was confirmed by immunohistochemical staining (FIG. 17E). Taken together, tissue expression of B7H1 also plays an important role in GVHD prevention in unconditioned recipients.

### Example 12

### Donor T Cell-Derived IFN-γ Played a Critical Role in Induction of Host Tissue Cell Expression of B7H1 and Prevention of GVHD

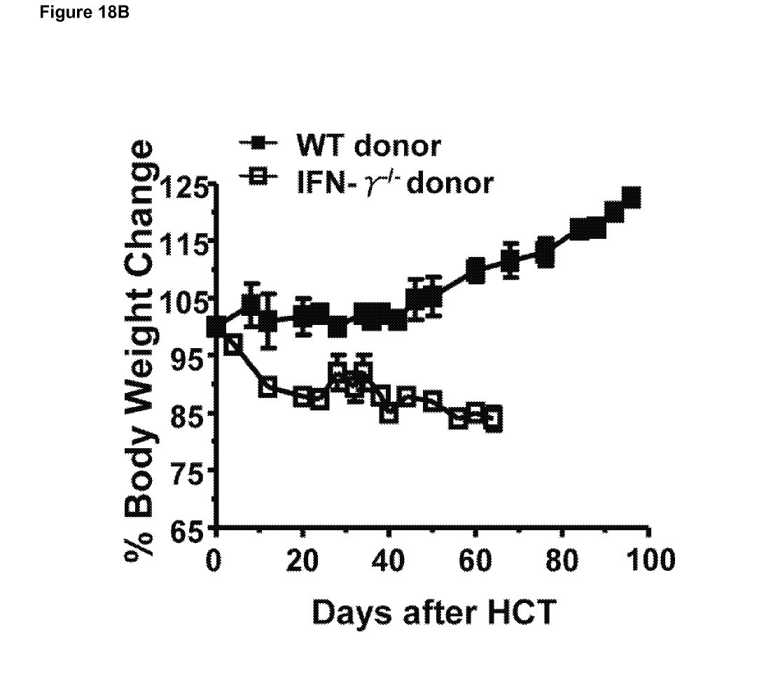

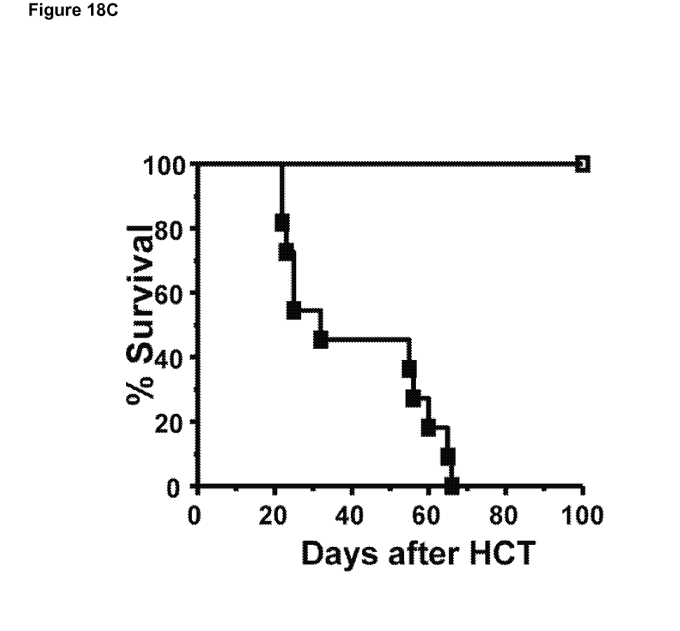

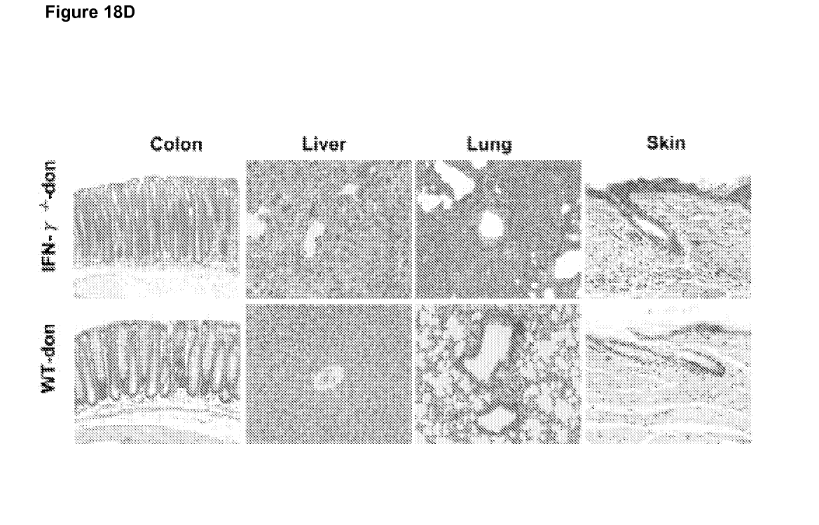

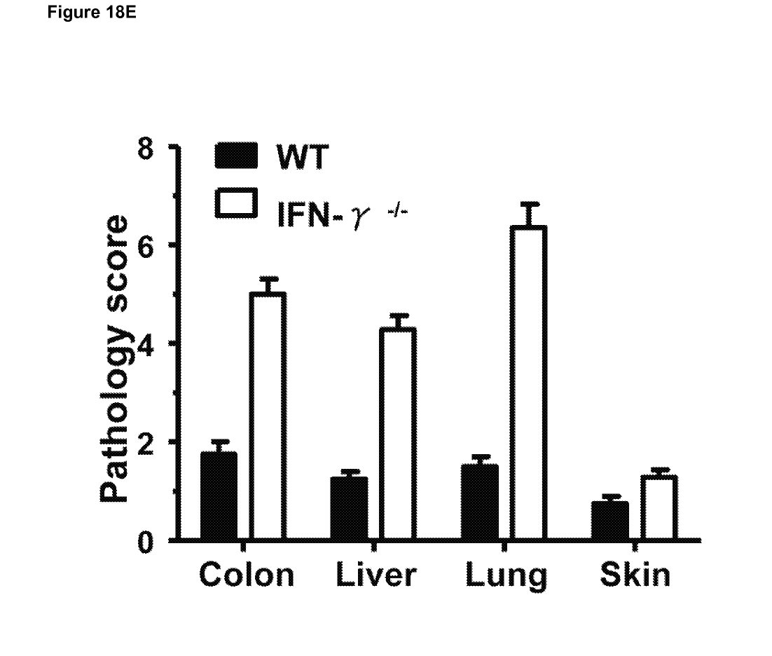

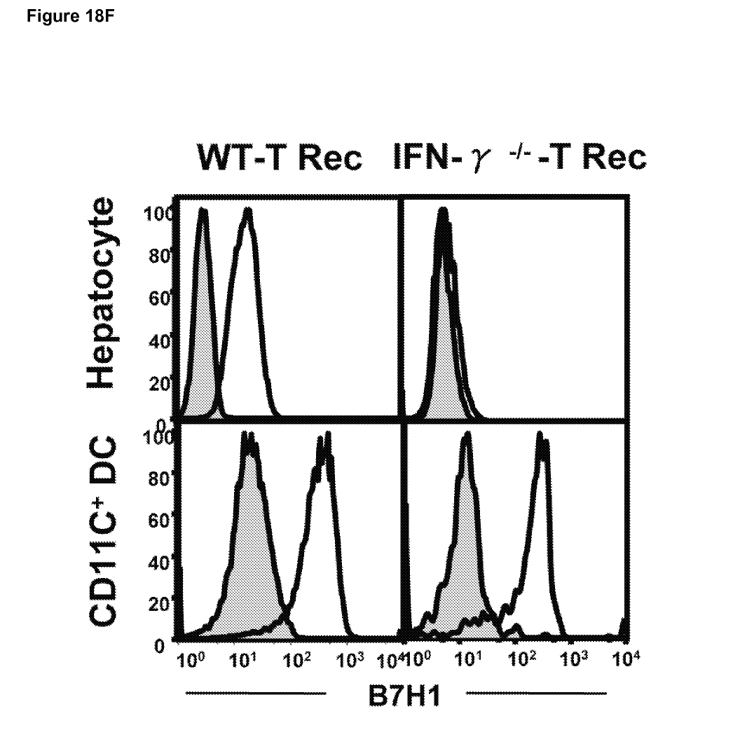

Tissue parenchymal cell expression of B7H1 is induced by IFN-γ, although the hematopoietic cells such as DCs constitutively express surface B7H1. Next, whether donor T cell-derived IFN-γ was required for induction of GVHD target tissue expression of B7H1 was tested. Accordingly, sorted CD8⁺ T cells (20×10⁶) from IFN-γ^{-/-} or WT C57BL/6 donors were co-injected with TCD-BM from WT C57BL/6 donors into Rag2^{-/-} recipients. While Rag2^{-/-} recipients given WT donor CD8⁺ T cells showed little signs of GVHD, the recipients given IFN-γ^{-/-} donor CD8⁺ T cells showed severe clinical GVHD (i.e. weight-loss and diarrhea), and all of them died within 60 days after HCT (P<0.01, FIG. 18A-C). Consistently, the latter recipients showed much more severe infiltration and tissue damage in colon, liver, lung and skin (P<0.01, FIGS. 18D & E). Furthermore, while the hepatocytes of recipients given WT donor CD8⁺ T cells expressed high levels of B7H1 after HCT, the hepatocytes of the recipients given IFN-γ^{-/-} donor CD8⁺ T cells expressed little B7H1 (FIG. 18F), although CD11c⁺ DC cells from both recipients expressed high level of B7H1. These results demonstrate that donor T-derived IFN-γ play an important role in induction of B7H1 expression on tissue parenchymal cells and that expression of B7H1 by tissue parenchymal cells but not hematopoietic cells played a critical role in prevention of GVHD. Accordingly, donor T-derived IFN-γ may be controlled such that IFN-γ is present and/or upregulated to produce sufficient B7H1 to prevent or reduce the severity of GVHD.

### Example 13

### Expression of B7H1 by Tissue Parenchymal Cells Played a Critical Role in GVHD Prevention

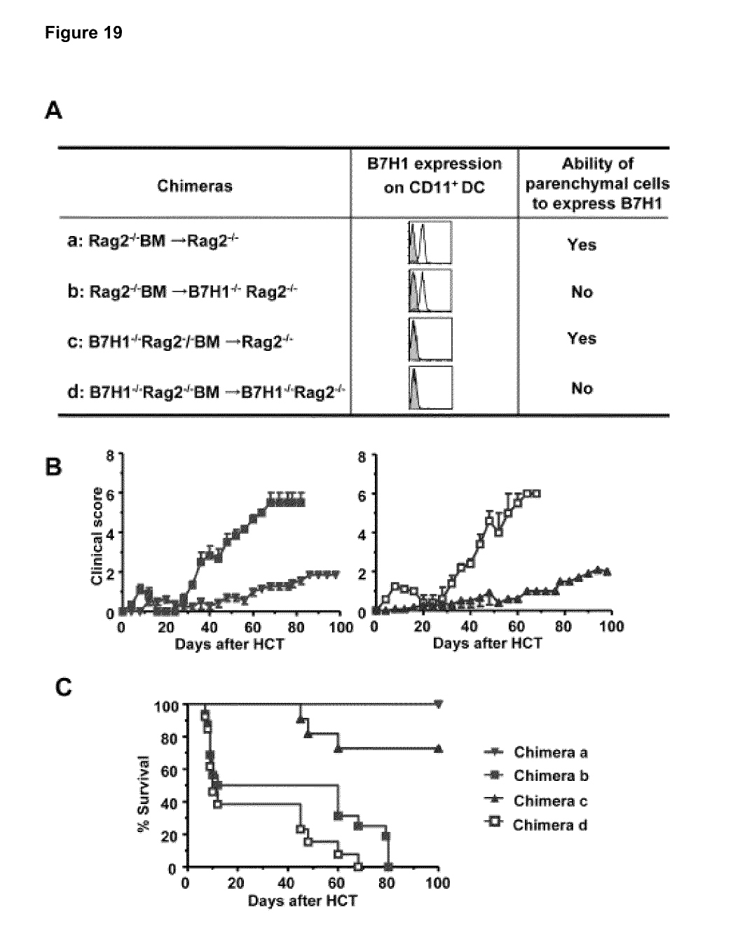

To distinguish the role of B7H1 expressed by tissue parenchymal cells and by tissue hematopoietic cells such as DCs in GVHD prevention, it was established that chimeric Rag2^{-/-} mice whose parenchymal cells were but hematopoietic cells were not able to express B7H1 by transplantation of B7H1^{-/-}Rag2^{-/-} BM cells into lethally irradiated Rag2^{-/-} mice (Chimera c in FIG. 19A). Conversely, it was established that chimeric B7H1^{-/-}Rag2^{-/-} mice whose parenchymal cells were not but hematopoietic cells were able to express B7H1 by transplantation of Rag2^{-/-}BM into lethally irradiated B7H1^{-/-}Rag2^{-/-} mice (Chimera b in FIG. 19A). The control mice were Rag2^{-/-} mice reconstituted with Rag2^{-/-} BM (Chimera a in FIG. 19A) and B7H1^{-/-}Rag2^{-/-} mice reconstituted with B7H1^{-/-}Rag2^{-/-} BM (Chimera d in FIG. 19A). Eight weeks after BM transfer, the induction of chimerism was confirmed by the B7H1 expression on CD11c⁺ cells in blood of the chimeras (FIG. 19A). Thereafter, all chimeras were transplanted with CD8⁺ T cells (20×10⁶) and TCD-BM from C57BL/6 donors.

While 100% of the Chimera a recipients given donor CD8⁺ T cells developed only mild signs of GVHD and all survived for more than 100 days, 100% of Chimera b recipients given donor CD8⁺ T cells developed severe GVHD and all died within 80 days (P<0.01, FIGS. 19B & C). Conversely, while 75% of Chimeras c recipients developed only mild signs of GVHD and survival for more than 100 days, 100% of Chimera d recipients developed severe clinical GVHD and died by 60 days after HCT (P<0.01, FIGS. 19 B & C). The GVHD severity and percentage of survival between Chimeras a recipients and Chimeras c recipients was slightly different (P=0.035, FIGS. 19 B & C), and the GVHD severity and percentage of survival between Chimeras b recipients and Chimeras d recipients were not significantly different (P=0.069, FIGS. 19 B & C). These results demonstrate that, B7H1 expression by host tissue parenchymal cells plays a critical role in GVHD prevention, although B7H1 expression by host hematopoietic cells also plays a minor role. Thus, GVHD may be prevented by maintaining sufficient levels of B7H1 in host tissue parenchymal cells.

### Example 14

### Tissue Expression of B7H1 Inhibited Expansion of Donor Memory T Cells in GVHD Target Tissues

To further explore the mechanisms whereby tissue expression of B7H1 prevented GVHD, in vivo BLI was first used to visualize the donor CD8⁺ T cell expansion in B7H1 sufficient Rag2^{-/-} and B7H1 deficient B7H1^{-/-}Rag2^{-/-} recipients. Accordingly, the recipients were injected with sorted donor CD8⁺ T (20×10⁶) from Luc⁺ C57BL/6 (CD45.2) donors and TCD-BM cells from congenic C57BL/6 (CD45.1) donors. In Rag2^{-/-} recipients, donor CD8⁺ T cells expanded slowly and maintained at low-level for up to 45 days after HCT. In contrast, although compared to Rag^{-/-} recipients, the donor CD8⁺ T cell expansion in B7H1^{-/-}Rag^{-/-} recipients was only slightly stronger 1˜10 days after HCT, their expansion was more rapidly thereafter and reached the first peak that was about 2-fold stronger than in Rag2^{-/-} recipients 13 days after HCT (P<0.01). Then, it subsided partially for about 2 weeks.

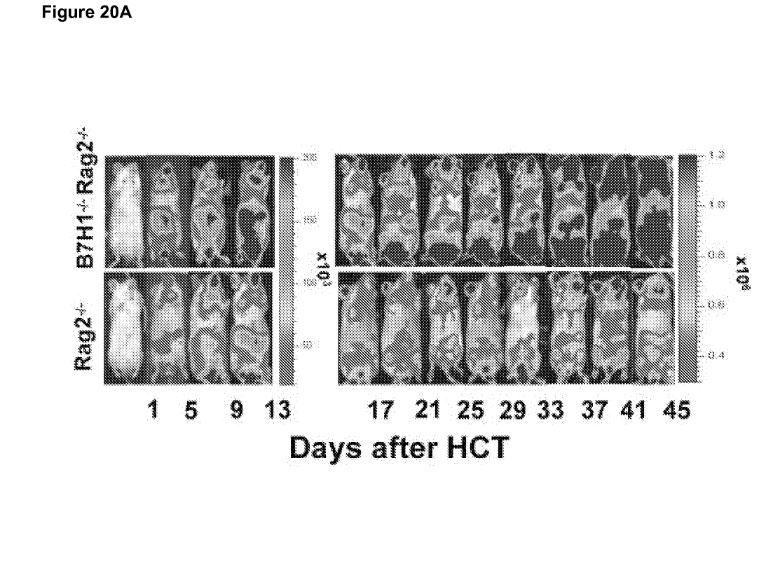

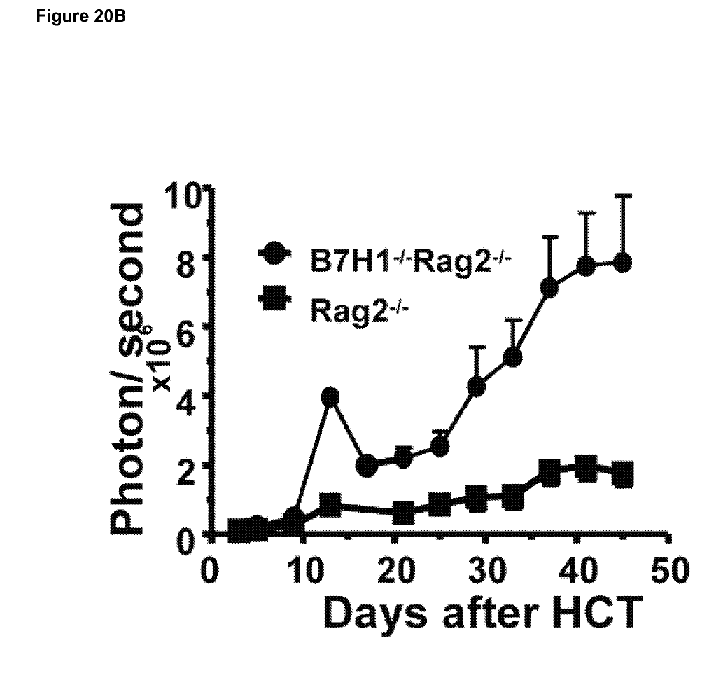

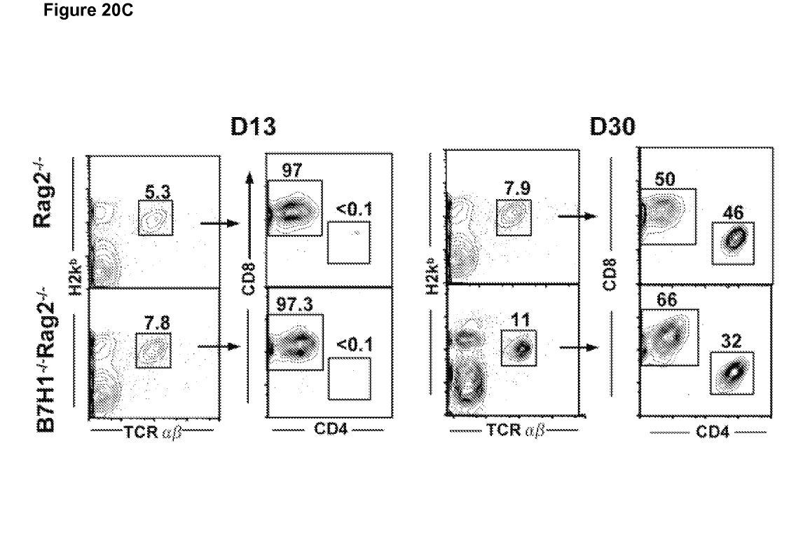

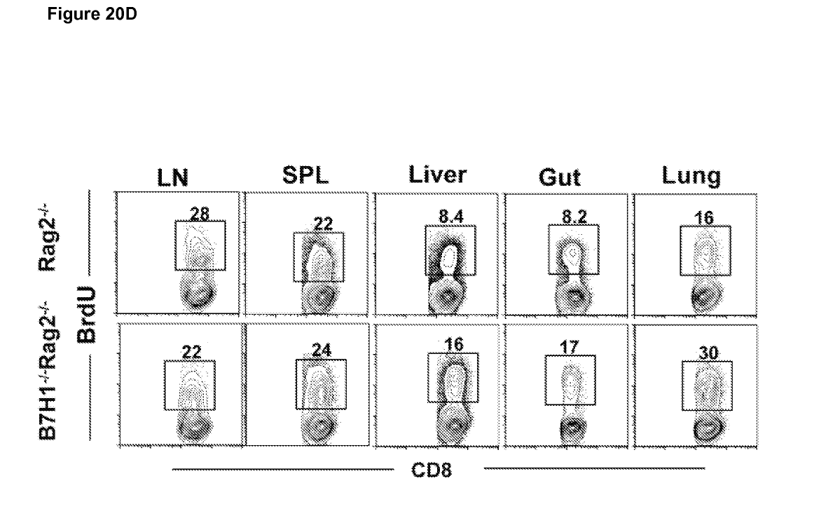

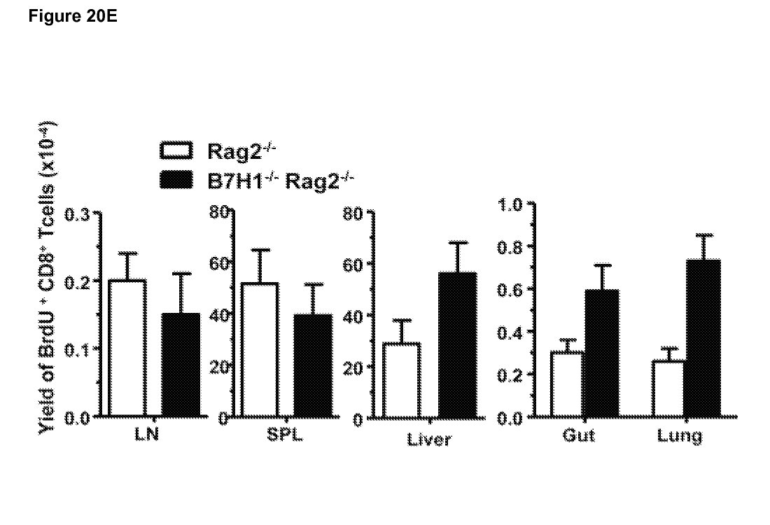

By approximately 30 days after HCT, donor CD8⁺ T cells in B7H1^{-/-}Rag2^{-/-} recipients expanded again, which was 4-fold stronger than in Rag2^{-/-} recipients (P<0.01, FIGS. 20 A & B). The second wave of donor CD8⁺ T expansion in B7H1^{-/-}Rag-2^{-/-} recipients was at least 2-fold stronger than the first one (P<0.01, FIGS. 20A & B). The second wave of expansion was associated with the presence of de novo developed donor-type CD4⁺ T cells (FIG. 20C). The BLI was not continued 45 days after HCT due to the severe GVHD and anesthesia related death in the group of B7H1^{-/-}Rag2^{-/-} recipients. Because almost all donor CD8⁺ T cells became CD62L^{lo}CD44^{hi} memory T cells 13 days after HCT, and it was demonstrated that parenchymal cell expression of B7H1 played a critical role in GVHD prevention, thus, the lack of the second wave expansion of donor CD8⁺ T cells in B7H1 sufficient Rag2^{-/-} recipients demonstrates that host parenchymal tissue expression of B7H1 inhibits donor memory T cell expansion.

Memory T cells can expand in lymph nodes, spleen, and tissues. To test whether the rapid expansion of donor T cells in B7H1^{-/-}Rag2^{-/-} recipients was in host lymphoid tissues (i.e. lymph node and spleen) or in GVHD target tissues, in vivo proliferation of donor T cells in spleen and lymph nodes was compared with that in GVHD target tissues of B7H1 sufficient Rag2^{-/-} or B7H1 deficient B7H1^{-/-}Rag2^{-/-} recipients by in vivo BrdU-labeling assays on days 5, 13, and 30 after HCT. The injected donor CD8⁺ T cells were distinguished from the de novo developed donor CD8⁺ T cells with anti-CD45.2 staining. 5 days after HCT, the percentage of donor Brdu⁺CD8⁺ T cells in B7H1^{-/-}Rag2^{-/-} recipients was about 30% higher than that in Rag-2^{-/-} recipients (P<0.05, and the yield in the former was 2-fold higher than that in the latter (P<0.01). However, there was no significant difference in percentage or yield in liver of the two groups, and the yield of both was very low, although majority of them were BrdU⁺. These results demonstrate that early after HCT, donor T cell proliferation takes place mainly in host lymphoid tissues, and host DC expression of B7H1 play an important role in limiting donor T expansion.

By 13 days after HCT, T cell proliferation in spleen was relatively reduced in both recipients, and the percentage and yield of donor Brdu⁺CD8⁺ T cells in spleen of both recipients was no longer significantly different. However, the percentage and yield of donor BrdU⁺CD8⁺ T cells in liver of B7H1−/−Rag-2−/− recipients appeared to be higher than Rag-2^{-/-} recipients. Furthermore, by 30 days after HCT, although T cell proliferation in lymph node and spleen was still similar in both recipients, T cell proliferation in GVHD target tissues including liver, gut and lung was more than 2-fold stronger in B7H1^{-/-}Rag-2^{-/-} recipients than in Rag2^{-/-} recipients as judged by the percentage and yield of donor BrdU⁺CD8⁺ T cells (P<0.01, FIGS. 20 D and E). These results demonstrate that late after HCT GVHD target tissue expression of B7H1 inhibits donor memory T cell proliferation. Use of B7H1 and other effective agents that suppress donor memory T cell proliferation for the prevention of GVHD in HCT recipients has been thus discovered.

### Example 15

### Radiation-Free Anti-CD3-Based Conditioning Regimen Might Better Maintain the Tissue Protective Mechanism Mediated by Parenchymal Tissue Expression of B7H1 Late after HCT

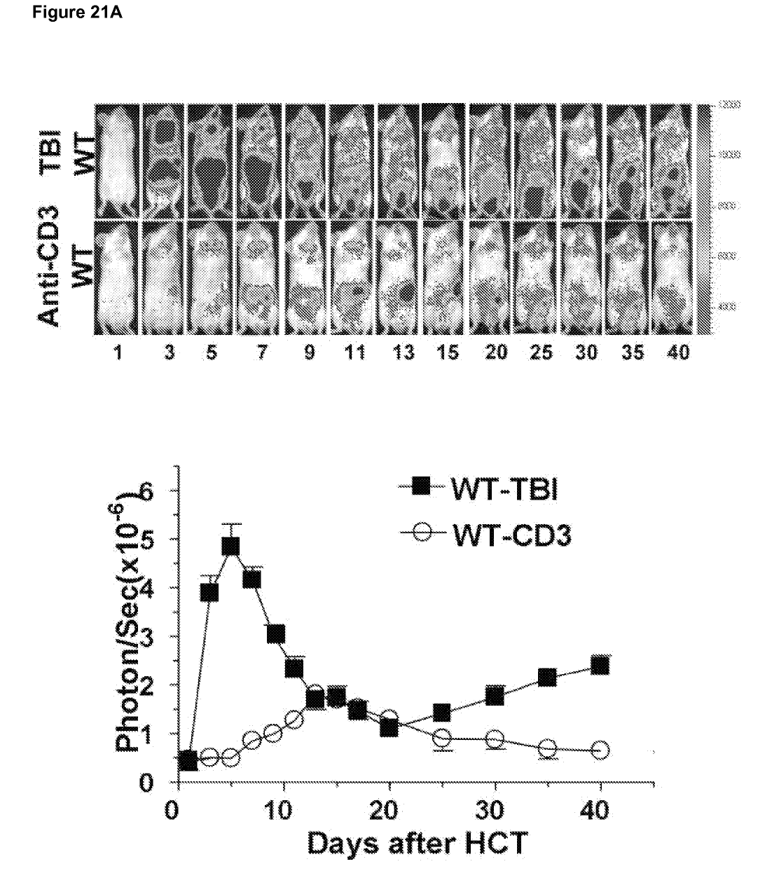

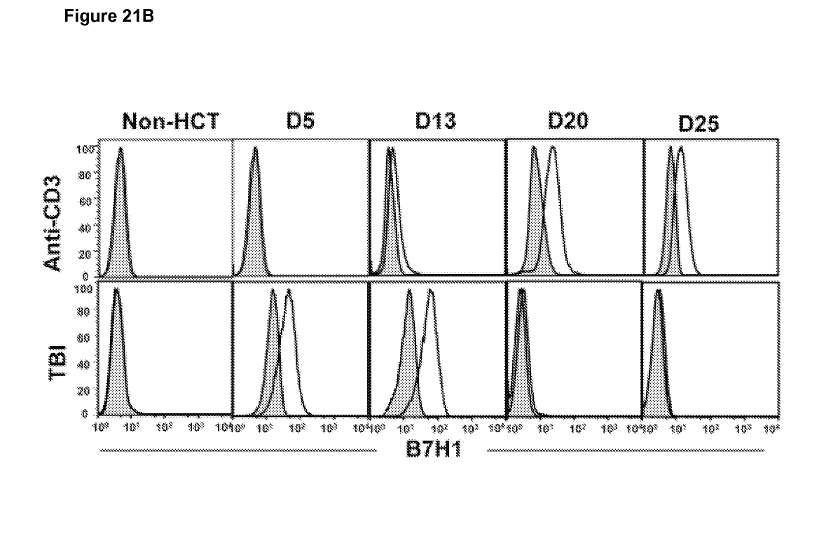

It was reported previously that donor CD8⁺ T showed two peaks of expansion in TBI-conditioned recipients but only a single small peak in anti-CD3-conditioned recipients. Next, whether the donor CD8⁺ T expansion peaks were associated with lack of tissue expression of B7H1 was tested. First, it was observed that, in TBI-conditioned recipients, donor CD8⁺ T cells showed first wave of expansion 3-15 days after HCT and second wave of expansion approximately 30 days after HCT. In contrast, donor CD8⁺ T cells in anti-CD3-conditioned recipients showed a weak expansion 3-15 days after HCT and no second wave of expansion was observed (FIG. 21A). Accordingly, the B7H1 expression by hepatocytes of anti-CD3 or TBI-conditioned recipients were compared on days 5, 13, 20, and 25 after HCT. In TBI-conditioned recipients, the hepatocytes expressed B7H1 on days 5 and 13 after HCT, but they no longer expressed B7H1 after day 20. In contrast, in anti-CD3-conditioned recipients, hepatocyte expression of B7H1 was gradually increased and reached the peak 20 days after HCT and the expression continued after day 25 (FIG. 21B). Combined with the fact that donor CD8⁺ T cells also showed the second wave expansion in non-TBI-conditioned B7H1^{-/-} recipients, these results demonstrate that the second wave expansion of donor CD8⁺ T (memory T) cells in TBI-conditioned recipients are associated with the lack of expression of B7H1 by host parenchymal cells. Thus, TBI-conditioning may lead to the damage of the B7H1 mediated tissue protective mechanism.

The examples disclosed herein are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention. All references cited herein are incorporated by reference as if fully set forth herein.

### REFERENCES

1. Agata Y, Kawasaki A, Nishimura H, et al. Expression of the PD-1 antigen on the surface of stimulated mouse T and B lymphocytes. Int Immunol. 1996; 8:765-772.
2. Appelbaum F R. Haematopoietic cell transplantation as immunotherapy. Nature. 2001; 411:385-389.
3. Beaty S R, Rose C E, Jr., Sung S S. Diverse and potent chemokine production by lung CD11 bhigh dendritic cells in homeostasis and in allergic lung inflammation. J. Immunol. 2007; 178:1882-1895.
4. Beilhack A, Schulz S, Baker J, et al. In vivo analyses of early events in acute graft-versus-host disease reveal sequential infiltration of T-cell subsets. Blood. 2005; 106:1113-1122.
5. Beilhack A, Schulz S, Baker J, et al. Prevention of acute graft-versus-host disease by blocking T-cell entry to secondary lymphoid organs. Blood. 2008; 111:2919-2928.
6. Blazar B R, Jenkins M K, Taylor P A, et al. Anti-CD3 epsilon F(ab′)2 fragments inhibit T cell expansion in vivo during graft-versus-host disease or the primary immune response to nominal antigen. J. Immunol. 1997; 159:5821-5833.
7. Blazar B R, Carreno B M, Panoskaltsis-Mortari A, et al. Blockade of programmed death-1 engagement accelerates graft-versus-host disease lethality by an IFN-gamma-dependent mechanism. J. Immunol. 2003; 171:1272-1277.
8. Burman A C, Banovic T, Kuns R D, et al. IFNgamma differentially controls the development of idiopathic pneumonia syndrome and GVHD of the gastrointestinal tract. Blood. 2007; 110:1064-1072.
9. Cao Y A, Wagers A J, Beilhack A, et al. Shifting foci of hematopoiesis during reconstitution from single stem cells. Proc Natl Acad Sci USA. 2004; 101:221-226.
10. Chakraverty R, Cote D, Buchli J, et al. An inflammatory checkpoint regulates recruitment of graft-versus-host reactive T cells to peripheral tissues. J Exp Med. 2006; 203:2021-2031.
11. Chakraverty R, Sykes M. The role of antigen-presenting cells in triggering graft-versus-host disease and graft-versus-leukemia. Blood. 2007; 110:9-17.
12. Chatenoud L, Bluestone JA. CD3-specific antibodies: a portal to the treatment of autoimmunity. Nat Rev Immunol. 2007; 7:622-632.
13. Chen L. Co-inhibitory molecules of the B7-CD28 family in the control of T-cell immunity. Nat Rev Immunol. 2004; 4:336-347.
14. Cooke K R, Kobzik L, Martin T R, et al. An experimental model of idiopathic pneumonia syndrome after bone marrow transplantation: I. The roles of minor H antigens and endotoxin. Blood. 1996; 88:3230-3239.
15. Cooke K R, Gerbitz A, Crawford J M, et al. LPS antagonism reduces graft-versus-host disease and preserves graft-versus-leukemia activity after experimental bone marrow transplantation. J Clin Invest. 2001; 107:1581-1589.
16. Coombes J L, Siddiqui K R, Arancibia-Carcamo C V, et al. A functionally specialized population of mucosal CD103⁺ DCs induces Foxp3+ regulatory T cells via a TGF-beta and retinoic acid-dependent mechanism. J Exp Med. 2007; 204:1757-1764.
17. Decalf J, Fernandes S, Longman R, et al. Plasmacytoid dendritic cells initiate a complex chemokine and cytokine network and are a viable drug target in chronic HCV patients. J Exp Med. 2007; 204:2423-2437.
18. Dong H, Zhu G, Tamada K, Flies D B, van Deursen J M, Chen L. B7-H1 determines accumulation and deletion of intrahepatic CD8(+) T lymphocytes. Immunity. 2004; 20:327-336.
19. Dong H, Strome S E, Salomao D R, et al. Tumor-associated B7-H1 promotes T-cell apoptosis: a potential mechanism of immune evasion. Nat Med. 2002; 8:793-800.
20. Drakes M L C S, Blanchard T G. Isolation and purification of colon lamina propria dendritic cells from mice with colitis. Cytotechnology. 2004; 46:151-161.
21. Duffner U A, Maeda Y, Cooke K R, et al. Host dendritic cells alone are sufficient to initiate acute graft-versus-host disease. J. Immunol. 2004; 172:7393-7398.
22. Duffner U, Lu B, Hildebrandt G C, et al. Role of CXCR3-induced donor T-cell migration in acute GVHD. Exp Hematol. 2003; 31:897-902.
23. Ferrara J, Antin. J. The Pathophysiology of Graft-vs.-Host Disease. In: Blume K G, Forman, S. J., and Appelbaum, F. R., ed. Thomas' Hematopoietic cell transplantation. Malden, Mass.: Blackwell Publishing Ltd; 2004:353-368.
24. Fife B T, Bluestone J A. Control of peripheral T-cell tolerance and autoimmunity via the CTLA-4 and PD-1 pathways. Immunol Rev. 2008; 224:166-182.
25. Giralt S. Reduced-intensity Conditioning Regimens for Hematologic Malignancies: What Have We Learned over the Last 10 Years? Hematology Am Soc Hematol Educ Program. 2005: 384-389.
26. Greenwald R J, Freeman G J, Sharpe A H. The B7 family revisited. Annu Rev Immunol. 2005; 23:515-548.
27. Haspot F, Fehr T, Gibbons C, et al. Peripheral deletional tolerance of alloreactive CD8 but not CD4 T cells is dependent on the PD-1/PD-L1 pathway. Blood. 2008; 112:2149-2155.
28. Hieshima K, Kawasaki Y, Hanamoto H, et al. CC chemokine ligands 25 and 28 play essential roles in intestinal extravasation of IgA antibody-secreting cells. J. Immunol. 2004; 173:3668-3675.
29. Hildebrandt G C, Corrion L A, Olkiewicz K M, et al. Blockade of CXCR3 receptor: ligand interactions reduces leukocyte recruitment to the lung and the severity of experimental idiopathic pneumonia syndrome. J. Immunol. 2004; 173:2050-2059.
30. Ishida M, Iwai Y, Tanaka Y, et al. Differential expression of PD-L1 and PD-L2, ligands for an inhibitory receptor PD-1, in the cells of lymphohematopoietic tissues. Immunol Lett. 2002; 84:57-62.
31. Iwata M, Hirakiyama A, Eshima Y, Kagechika H, Kato C, Song S Y. Retinoic acid imprints gut-homing specificity on T cells. Immunity. 2004; 21:527-538.
32. Jaksch M, Remberger M, Mattsson J. Increased gene expression of chemokine receptors is correlated with acute graft-versus-host disease after allogeneic stem cell transplantation. Biol Blood Marrow Transplant. 2005; 11:280-287.
33. Johansson-Lindbom B, Svensson M, Pabst O, et al. Functional specialization of gut CD103+ dendritic cells in the regulation of tissue-selective T cell homing. J Exp Med. 2005; 202:1063-1073.
34. Johansson-Lindbom B, Svensson M, Wurbel M A, Malissen B, Marquez G, Agace W. Selective generation of gut tropic T cells in gut-associated lymphoid tissue (GALT): requirement for GALT dendritic cells and adjuvant. J Exp Med. 2003; 198:963-969.
35. Johnson B D, McCabe C, Hanke C A, Truitt R L. Use of anti-CD3 epsilon F(ab′)2 fragments in vivo to modulate graft-versus-host disease without loss of graft-versus-leukemia reactivity after MHC-matched bone marrow transplantation. J. Immunol. 1995; 154:5542-5554.
36. Kaplan D H, Anderson B E, McNiff J M, Jain D, Shlomchik M J, Shlomchik W D. Target antigens determine graft-versus-host disease phenotype. J. Immunol. 2004; 173:5467-5475.
37. Keir M E, Liang S C, Guleria I, et al. Tissue expression of PD-L1 mediates peripheral T cell tolerance. J Exp Med. 2006; 203:883-895.
38. Kim T D, Terwey T H, Zakrzewski J L, et al. Organ-derived dendritic cells have differential effects on alloreactive T cells. Blood. 2008; 111:2929-2940.
39. Kim Y M, Sachs T, Asavaroengchai W, Bronson R, Sykes M. Graft-versus-host disease can be separated from graft-versus-lymphoma effects by control of lymphocyte trafficking with FTY720. J Clin Invest. 2003; 111:659-669.
40. Li N, Chen Y, He W, et al. Anti-CD3 preconditioning separates GVL from GVHD via modulating host dendritic cell and donor T-cell migration in recipients conditioned with TBI. Blood. 2009; 113:953-962.
41. Li N, Zhao D, Kirschbaum M, et al. HDAC inhibitor reduces cytokine storm and facilitates induction of chimerism that reverses lupus in anti-CD3 conditioning regimen. Proc Natl Acad Sci USA. 2008; 105:4796-4801.
42. Liang Y, Huang T, Zhang C, et al. Donor CD8+ T cells facilitate induction of chimerism and tolerance without GVHD in autoimmune NOD mice conditioned with anti-CD3 mAb. Blood. 2005; 105:2180-2188.
43. Mapara M Y, Leng C, Kim Y M, et al. Expression of chemokines in GVHD target organs is influenced by conditioning and genetic factors and amplified by GVHR. Biol Blood Marrow Transplant. 2006; 12:623-634.
44. Marzo A L, Vezys V, Klonowski K D, et al. Fully functional memory CD8 T cells in the absence of CD4 T cells. J. Immunol. 2004; 173:969-975.
45. Merad M, Hoffmann P, Ranheim E, et al. Depletion of host Langerhans cells before transplantation of donor alloreactive T cells prevents skin graft-versus-host disease. Nat Med. 2004; 10:510-517.
46. Mora J R, Cheng G, Picarella D, Briskin M, Buchanan N, von Andrian U H. Reciprocal and dynamic control of CD8 T cell homing by dendritic cells from skin- and gut-associated lymphoid tissues. J Exp Med. 2005; 201:303-316.
47. Mucida D, Park Y, Kim G, et al. Reciprocal TH17 and regulatory T cell differentiation mediated by retinoic acid. Science. 2007; 317:256-260.
48. Muhlbauer M, Fleck M, Schutz C, et al. PD-L1 is induced in hepatocytes by viral infection and by interferon-alpha and -gamma and mediates T cell apoptosis. J. Hepatol. 2006; 45:520-528.
49. Murai M, Yoneyama H, Harada A, et al. Active participation of CCR5(+)CD8(+) T lymphocytes in the pathogenesis of liver injury in graft-versus-host disease. J Clin Invest. 1999; 104:49-57.
50. Muraille E, Andris F, Pajak B, et al. Downregulation of antigen-presenting cell functions after administration of mitogenic anti-CD3 monoclonal antibodies in mice. Blood. 1999; 94:4347-4357.
51. Murphy W J, Welniak L A, Taub D D, et al. Differential effects of the absence of interferon-gamma and IL-4 in acute graft-versus-host disease after allogeneic bone marrow transplantation in mice. J Clin Invest. 1998; 102:1742-1748.
52. Nakazawa A, Dotan I, Brimnes J, et al. The expression and function of costimulatory molecules B7H and B7-H1 on colonic epithelial cells. Gastroenterology. 2004; 126:1347-1357.
53. Nishimura H, Okazaki T, Tanaka Y, et al. Autoimmune dilated cardiomyopathy in PD-1 receptor-deficient mice. Science. 2001; 291:319-322.
54. Nishimura H, Nose M, Hiai H, Minato N, Honjo T. Development of lupus-like autoimmune diseases by disruption of the PD-1 gene encoding an ITIM motif-carrying immunoreceptor. Immunity. 1999; 11:141-151.
55. Ohl L, Mohaupt M, Czeloth N, et al. CCR7 governs skin dendritic cell migration under inflammatory and steady-state conditions. Immunity. 2004; 21:279-288.
56. Olson T S, Ley K. Chemokines and chemokine receptors in leukocyte trafficking. Am J Physiol Regul Integr Comp Physiol. 2002; 283:R7-28.
57. Panoskaltsis-Mortari A, Price A, Hermanson J R, et al. In vivo imaging of graft-versus-host-disease in mice. Blood. 2004; 103:3590-3598.
58. Perruche S, Zhang P, Liu Y, Saas P, Bluestone J A, Chen W. CD3-specific antibody-induced immune tolerance involves transforming growth factor-beta from phagocytes digesting apoptotic T cells. Nat Med. 2008; 14:528-535.
59. Reddy P, Maeda Y, Liu C, Krijanovski 01, Korngold R, Ferrara J L. A crucial role for antigen-presenting cells and alloantigen expression in graft-versus-leukemia responses. Nat Med. 2005; 11:1244-1249.
60. Reiss Y, Proudfoot A E, Power C A, Campbell J J, Butcher E C. CC chemokine receptor (CCR)4 and the CCR10 ligand cutaneous T cell-attracting chemokine (CTACK) in lymphocyte trafficking to inflamed skin. J Exp Med. 2001; 194:1541-1547.
61. Sackstein R. A revision of Billingham's tenets: the central role of lymphocyte migration in acute graft-versus-host disease. Biol Blood Marrow Transplant. 2006; 12:2-8.
62. Schoop R, Wahl P, Le Hir M, Heemann U, Wang M, Wuthrich R P. Suppressed T-cell activation by IFN-gamma-induced expression of PD-L1 on renal tubular epithelial cells. Nephrol Dial Transplant. 2004; 19:2713-2720.
63. Shlomchik W D. Graft-versus-host disease. Nat Rev Immunol. 2007; 7:340-352.
64. Shlomchik W D, Couzens M S, Tang C B, et al. Prevention of graft versus host disease by inactivation of host antigen-presenting cells. Science. 1999; 285:412-415.
65. Shlomchik W D, Lee S J, Couriel D, Pavletic S Z. Transplantation's greatest challenges: advances in chronic graft-versus-host disease. Biol Blood Marrow Transplant. 2007; 13:2-10.
66. Sigmundsdottir H, Pan J, Debes G F, et al. DCs metabolize sunlight-induced vitamin D3 to ‘program’ T cell attraction to the epidermal chemokine CCL27. Nat Immunol. 2007; 8:285-293.
67. Slavin S, Nagler A, Naparstek E, et al. Nonmyeloablative stem cell transplantation and cell therapy as an alternative to conventional bone marrow transplantation with lethal cytoreduction for the treatment of malignant and nonmalignant hematologic diseases. Blood. 1998; 91:756-763.
68. Stenstad H, Svensson M, Cucak H, Kotarsky K, Agace W W. Differential homing mechanisms regulate regionalized effector CD8alphabeta+ T cell accumulation within the small intestine. Proc Natl Acad Sci USA. 2007; 104:10122-10127.
69. Sullivan K M. Graft-versus-host disease. In: Blume K G, Forman, S. J., and Appelbaum, F. R., ed. Thomas' Hematopoietic cell transplantation. Malden, Mass.: Blackwell Publishing Ltd; 2004:635-664.
70. Sun J C, Williams M A, Bevan M J. CD4+ T cells are required for the maintenance, not programming, of memory CD8+ T cells after acute infection. Nat Immunol. 2004; 5:927-933.
71. Terwey T H, Kim T D, Kochman A A, et al. CCR2 is required for CD8-induced graft-versus-host disease. Blood. 2005; 106:3322-3330.
72. Teshima T, Ordemann R, Reddy P, et al. Acute graft-versus-host disease does not require alloantigen expression on host epithelium. Nat Med. 2002; 8:575-581.
73. Tietz W, Allemand Y, Borges E, et al. CD4+ T cells migrate into inflamed skin only if they express ligands for E- and P-selectin. J. Immunol. 1998; 161:963-970.
74. Turnbull E L, Yrlid U, Jenkins C D, Macpherson G G. Intestinal dendritic cell subsets: differential effects of systemic TLR4 stimulation on migratory fate and activation in vivo. J. Immunol. 2005; 174:1374-1384.
75. Varona R, Cadenas V, Gomez L, Martinez A C, Marquez G. CCR6 regulates CD4+ T-cell-mediated acute graft-versus-host disease responses. Blood. 2005; 106:18-26.
76. Wakim L M, Waithman J, van Rooijen N, Heath W R, Carbone F R. Dendritic cell-induced memory T cell activation in nonlymphoid tissues. Science. 2008; 319:198-202.
77. Webster A C, Pankhurst T, Rinaldi F, Chapman J R, Craig J C. Monoclonal and polyclonal antibody therapy for treating acute rejection in kidney transplant recipients: a systematic review of randomized trial data. Transplantation. 2006; 81:953-965.
78. Welniak L A, Blazar B R, Murphy W J. Immunobiology of allogeneic hematopoietic stem cell transplantation. Annu Rev Immunol. 2007; 25:139-170.
79. Wysocki C A, Burkett S B, Panoskaltsis-Mortari A, et al. Differential roles for CCR5 expression on donor T cells during graft-versus-host disease based on pretransplant conditioning. J. Immunol. 2004; 173:845-854.
80. Wysocki C A, Panoskaltsis-Mortari A, Blazar B R, Serody J S. Leukocyte migration and graft-versus-host disease. Blood. 2005; 105:4191-4199.
81. Yamazaki T, Akiba H, Iwai H, et al. Expression of programmed death 1 ligands by murine T cells and APC. J. Immunol. 2002; 169:5538-5545.
82. Yang Y G, Dey B R, Sergio J J, Pearson D A, Sykes M. Donor-derived interferon gamma is required for inhibition of acute graft-versus-host disease by interleukin 12. J Clin Invest. 1998; 102:2126-2135.
83. Yang F, Huang X, Yi T, Yen Y, Moore D D, Huang W. Spontaneous development of liver tumors in the absence of the bile acid receptor farnesoid X receptor. Cancer Res. 2007; 67:863-867.
84. Yi T, Zhao D, Lin C L, et al. Absence of donor Th17 leads to augmented Th1 differentiation and exacerbated acute graft-versus-host disease. Blood. 2008; 112:2101-2110.
85. Youngnak-Piboonratanakit P, Tsushima F, Otsuki N, et al. The expression of B7-H1 on keratinocytes in chronic inflammatory mucocutaneous disease and its regulatory role. Immunol Lett. 2004; 94:215-222.
86. Yu X Z, Albert M H, Martin P J, Anasetti C. CD28 ligation induces transplantation tolerance by IFN-gamma-dependent depletion of T cells that recognize alloantigens. J Clin Invest. 2004; 113:1624-1630.
87. Zeng D, Lan F, Hoffmann P, Strober S. Suppression of graft-versus-host disease by naturally occurring regulatory T cells. Transplantation. 2004; 77:S9-S11.
88. Zeng D, Hoffmann P, Lan F, Huie P, Higgins J, Strober S. Unique patterns of surface receptors, cytokine secretion, and immune functions distinguish T cells in the bone marrow from those in the periphery: impact on allogeneic bone marrow transplantation. Blood. 2002; 99:1449-1457.
89. Zhang Y, Shlomchik W D, Joe G, et al. APCs in the liver and spleen recruit activated allogeneic CD8+ T cells to elicit hepatic graft-versus-host disease. J. Immunol. 2002; 169:7111-7118.
90. Zhang C, Todorov I, Zhang Z, et al. Donor CD4+ T and B cells in transplants induce chronic graft-versus-host disease with autoimmune manifestations. Blood. 2006; 107:2993-3001.
91. Zhang C, Lou J, Li N, et al. Donor CD8+ T cells mediate graft-versus-leukemia activity without clinical signs of graft-versus-host disease in recipients conditioned with anti-CD3 monoclonal antibody. J. Immunol. 2007; 178:838-850.
92. Zhang C, Todorov I, Lin C L, et al. Elimination of insulitis and augmentation of islet beta cell regeneration via induction of chimerism in overtly diabetic NOD mice. Proc Natl Acad Sci USA. 2007; 104:2337-2342.
93. Zhao D, Zhang C, Yi T, et al. In vivo-activated CD103+CD4+ regulatory T cells ameliorate ongoing chronic graft-versus-host disease. Blood. 2008; 112:2129-2138.

We claim:

1. A method of treating a hematological malignancy in a subject undergoing hematopoietic cell transplant (HCT), comprising administering a therapeutically effective amount of a CD3 modulator to the subject in advance of the HCT.

2. The method of claim 1, wherein the CD3 modulator is a CD3 agonist.

3. The method of claim 2, wherein the CD3 agonist is an anti-CD3 antibody or therapeutically effective fragment thereof or a small molecule.

4. The method of claim 3, wherein the antibody or therapeutically effective fragment thereof is a monoclonal antibody.

5. The method of claim 1, wherein the subject undergoes irradiation after administration of the CD3 modulator and before HCT.

6. The method of claim 5, wherein B7H1 is activated in the host tissues of the subject prior to the irradiation.

7. The method of claim 1, wherein the CD3 modulator is administered two weeks to two days in advance of HCT.

8. The method of claim 1, wherein the CD3 modulator is administered intravenously or orally.

9. The method of claim 1, wherein the hematological malignancy is graft versus host disease.

10. The method of claim 1, wherein the CD3 modulator is an anti-CD3 antibody, and the antibody is administered in conjunction with anti-CD28.

11. A method of preserving graft versus leukemia effect while preventing graft versus host disease in a subject preparing for an allogeneic hematopoietic cell transplant (HCT) comprising preconditioning the subject by administrating to the subject a therapeutically effective amount of a CD3 agonist before the subject undergoes HCT.

12. The method of claim 11, wherein the CD3 agonist is an anti-CD3 antibody or therapeutically effective fragment thereof.

13. The method of claim 12, wherein the anti-CD3 antibody or therapeutically effective fragment thereof is monoclonal.

14. The method of claim 12, wherein the anti-CD3 antibody or therapeutically effective fragment thereof is polyclonal.

15. The method of claim 11, wherein the CD3 agonist is administered in the time period of two weeks to two days before HCT.

16. The method of claim 11, wherein the CD3 agonist is administered intravenously in a pharmaceutically acceptable carrier.

17. The method of claim 11, wherein the subject undergoes one or more sessions of irradiation before receiving the HCT.

18. A method of preventing graft versus host disease in a subject undergoing allogeneic cell transplantation comprising administering a therapeutically effective amount of an anti-CD3 monoclonal antibody to the subject in advance of the cell transplantation.

19. The method of claim 18, wherein the method further comprises inducing or upregulating expression of B7H1 in the tissues of the subject before and/or after the cell transplantation.

20. The method of claim 18, wherein the antibody is administered intravenously in a pharmaceutically acceptable carrier.
